(19) United States
(12) Patent Application Publication
(10) Pub. No.: US 2016/0361255 A1
(43) Pub. Date: Dec. 15, 2016

(54) Peptide Nanotubes for Slow Release of Drugs at Peripheral Nerve Injury

(71) Applicant: The University of Toledo, Toledo, OH (US)

(72) Inventors: Joshua Park, Toledo, OH (US); Dong Shik Kim, Toledo, OH (US); Nicholas Chiaia, Toledo, OH (US)

(73) Assignee: The University of Toledo, Toledo, OH (US)

(21) Appl. No.: 15/177,672

(22) Filed: Jun. 9, 2016

Related U.S. Application Data
(60) Provisional application No. 62/175,018, filed on Jun. 12, 2015.

(51) Int. Cl. A61K 9/00; A61K 31/715; A61K 47/18

(52) U.S. Cl. CPC A61K 9/0092; A61K 47/183; A61K 31/715

(57) ABSTRACT

Described herein are materials and methods useful for the treatment of neurodegenerative disorders in a subject. Materials described herein include polysaccharide digestive products resulting from the enzymatic hydrolysis of low acyl gellan gum.

### RELATED APPLICATIONS

This application claims priority to U.S. Provisional Application Ser. No. 62/175,018 filed under 35 U.S.C. §111(b) on Jun. 12, 2015, the disclosure of which is incorporated herein by reference in its entirety for all purposes.

### BACKGROUND OF THE INVENTION

Alzheimer's disease (AD) is a progressive degenerative disorder of the brain that begins with memory impairment and eventually progresses to dementia, physical impairment, and death. Approximately 4.5 million people in the United States suffer from AD, costing over $100 billion annually. During AD development, oxidative stress significantly increases inside the brain, resulting in production of excessive free reactive radicals, such as reactive oxygen intermediates (ROI: superoxide anion and hydrogen peroxide) and 4-hydroxynonenal (4-HNE), a lipid peroxide. Amyloid β peptide (Aβ₄₂) also increases oxidative stress by damaging the mitochondria, resulting in generation of free radicals. Cortical and hippocampal neurons exposed to Aβ₄₂ begin neurite atrophy and then undergo apoptosis.

Parkinson's disease (PD) affects nearly 1 million Americans and is the second leading neurodegenerative disease in the United States. Parkinson's disease is a result of chronic progressive degeneration of neurons, the cause of which has not yet completely been clarified. While the primary cause of Parkinson disease is not known, it is characterized by the preferential loss of dopaminergic neurons in the substantia nigra and subsequent loss of dopamine in the striatum. Loss of dopamine in the striatum results in resting tremor, bradykinesia, rigidity and postural instability.

Remedies are needed that not only influence dopaminergic transmission and alleviate the symptoms of Parkinson's disease in advanced stages, but also reverse, prevent or at least significantly delay the extinction of dopaminergic neurons in the early, to a great extent motor-asymptomatic, Parkinson stages. However, the short half-life and blood-brain barrier (BBB) impermeability of protein-based neurotrophic factors limit their clinical uses. Thus, there is a need for an alternative neurotrophic agent that overcomes those shortcomings for better treatment of PD.

### SUMMARY OF THE INVENTION

Described herein are materials and methods useful for the treatment of neurodegenerative disorders in a subject. Materials described herein include polysaccharide digestive products resulting from the enzymatic hydrolysis of low acyl gellan gum.

In a particular embodiment described herein is a method of treating a degenerative neurological disorder comprising administering to a subject in need of such treatment, an effective amount of a low acyl gellan gum (LA-GAGR) cleavage product, thereby treating the degenerative neurological disorder.

In another particular embodiment provided herein are methods wherein the degenerative neurological disorder is selected from the group consisting of Alzheimer's disease, Parkinson's disease, multiple sclerosis, amyotrophic lateral sclerosis, spinal cord injury, brain trauma, and peripheral nerve injury.

In another particular embodiment provided herein are methods wherein the degenerative neurological disorder is Alzheimer's disease or Parkinson's disease.

In another particular embodiment provided herein are methods wherein the degenerative neurological disorder is amyotrophic lateral sclerosis.

In another particular embodiment provided herein are methods wherein the degenerative neurological disorder is a spinal cord injury.

In another embodiment provided herein are methods wherein the degenerative neurological disorder is a peripheral nerve injury.

In yet other embodiments provided herein are methods wherein the subject is a human.

In certain embodiments provided herein are methods wherein the effective amount of LA-GAGR cleavage product is administered in combination with a pharmaceutically acceptable carrier thereof.

Also provided herein are methods wherein the LA-GAGR cleavage product is midi-GAGR, wherein midi-GAGR has a molecular weight of about 4,775 g/mole.

Also provided herein are methods wherein the LA-GAGR cleavage product is mini-GAGR, wherein mini-GAGR has a molecular weight of about 718 g/mole.

Further provided is a composition comprising nanotubes assembled from a monomer solution comprising a diphenylalanine peptide, wherein a digestion product of low acyl gellan gum (LA-GAGR) is encapsulated in the nanotubes. In certain embodiments, the digestion product comprises midi-GAGR or mini-GAGR. In certain embodiments, the diphenylalanine peptide is dissolved in 1,1,1,3,3,3-hexafluoro-2-propanol. In certain embodiments, wherein the monomer solution is present in water at a concentration of about 2 mg/mL.

Further provided is a method of inducing nerve regeneration and/or treating peripheral nerve injury, the method comprising the steps of encapsulating midi-GAGR in peptide nanotubes to produce midi-GAGR-encapsulated nanotubes, and administering an effective amount of midi-GAGR-encapsulated nanotubes to a patient in need thereof to induce nerve regeneration. In certain embodiments, the peptide nanotubes comprise a diphenylalanine peptide dissolved in 1,1,1,3,3,3-hexafluoro-2-propanol. In certain embodiments, the administration is via sustained release of the midi-GAGR from the midi-GAGR-encapsulated nanotubes.

Further provided is a method of inducing nerve regeneration and/or treating peripheral nerve injury, the method comprising the steps of encapsulating mini-GAGR in peptide nanotubes to produce mini-GAGR-encapsulated nanotubes, and administering an effective amount of mini-GAGR-encapsulated nanotubes to a patient in need thereof to induce nerve regeneration. In certain embodiments, the peptide nanotubes comprise a diphenylalanine peptide dissolved in 1,1,1,3,3,3-hexafluoro-2-propanol. In certain embodiments, the administering is via sustained release of the mini-GAGR from the mini-GAGR-encapsulated nanotubes.

Further provided is a method of inducing nerve regeneration and/or treating peripheral nerve injury, the method comprising administering an effective amount of midi-GAGR or mini-GAGR to a subject in need thereof to induce nerve regeneration.

Further provided is a method of making peptide nanotubes, the method comprising the steps of dissolving a diphenylalanine peptide monomer in a highly volatile solvent to produce a monomer solution, diluting the monomer solution in water to cause self-assembly of peptide nanotubes, and drying the peptide nanotubes. In certain embodiments, the highly volatile solvent comprises 1,1,1,3,3,3-hexafluoro-2-propanol. In certain embodiments, the peptide nanotubes are encapsulated with a digestion product of low acyl gellan gum (LA-GAGR). Also provided is the product of the method.

### BRIEF DESCRIPTION OF THE DRAWINGS

The patent or application file contains at least one drawing executed in color. Copies of this patent or patent application publication with color drawings) will be provided by the Office upon request and payment of the necessary fee.

FIG. 1: Low acyl gellan gum (LA-GAGR). Drawing of the chemical structure of LA-GAGR ([D-Glc(β1→4)D-GlcA(β1→4)D-Glc(β1→4)L-Rha(α1→3]n). Arrows point to either α- or β-glucosidic bonds. Enzymatic decomposition of LA-GAGR by α(1→3)-glucosidase results in cleavage products having approximate molecular weights of either 4,755 g/mole (48 hour digestion) or 718 g/mole (72 hour digestion).

FIG. 2: Rheometer measurement of low acyl gellan gum (LA-GAGR). LA-GAGR shows an average molecular weight of approximately 99,639 g/mole.

FIG. 3: Rheometer measurement of 24 hour LA-GAGR digestion product. After 24 hours of enzymatic hydrolysis, the average molecular weight of LA-GAGR digestion product was reduced to approximately 30,245 g/mole. The three peaks shown indicate that α(1→3)-glucosidase breaks down LA-GAGR in multiple steps.

FIG. 4: Rheometer measurement of 48 hour LA-GAGR digestion product, midi-GAGR. 48 h enzymatic digestion of LA-GAGR yielded a product of approximately 4,775 g/mole (midi-GAGR). The single narrow peak shown indicates that 48 h digestion yielded a single product of an approximate equivalent length (molecular weight).

FIG. 5: Rheometer measurement of 72 h LA-GAGR digestion product, mini-GAGR. 72 h of enzymatic digestion of LA-GAGR yielded a product of approximately 718 g/mole (mini-GAGR).

FIGS. 6A-6D: Midi-GAGR protects mouse embryonic cortical neurons from the neurotoxicity of amyloid β peptide. Primary cortical neurons were treated with mock or 1 μM of dextran (FIG. 6A), alginate, high acyl gellan gum or midi-GAGR (FIG. 6B) for 6 h prior to the treatment with 10 μM amyloid β peptide for 48 h. The viability of neurons was assessed using LIVE/DEAD Viability/Cytotoxicity Assay Kit. Dead cells appear red and live cells appear green.

FIG. 6C: Midi-GAGR protects mouse embryonic cortical neurons from the neurotoxicity of amyloid β peptide. Depicted is a bar graph showing the percent of live cells (n>200 cells per condition×3, Mean±SEM).

FIG. 6D: Midi-GAGR protects mouse embryonic cortical neurons from the neurotoxicity of amyloid β peptide. Depicted is a flow chart summarizing the treatment protocol of primary cortical neurons used to generate FIGS. 6A-6C.

FIGS. 7A-7C: Midi-GAGR has neuroprotective effects on differentiated PC12 cells (into dopaminergic neurons). PC12 cells were differentiated in 100 ng/ml NGF, treat with mock (FIG. 7A), midi-GAGR (FIG. 7B), dextran, or alginate for 6 h, and then with 6-OHDA for 24 h. Dead cells in red and live cells in green.

FIG. 7C: Midi-GAGR has neuroprotective effects on differentiated PC12 cells (into dopaminergic neurons). Depicted is a bar graph showing the percent of dead cells (n>800 cells per condition×3, Mean±SEM).

FIGS. 8A-8C: Midi-GAGR has neuroprotective effects on mouse embryonic cortical neurons. Primary cortical neurons were treated with mock (FIG. 8A) or 1 μM of midi-GAGR (FIG. 8B), dextran, alginate, or high acyl gellan gum for 6 h prior to the treatment with amyloid β peptide for 24 h. Dead cells appear red and live cells appear green.

FIG. 8C: Midi-GAGR has neuroprotective effects on mouse embryonic cortical neurons. Depicted is a bar graph showing the percent of live cells (n>200 cells per condition×3, Mean±SEM).

FIG. 9A: Midi-GAGR increases the levels of phosphorylated CREB in the nucleus. Differentiated PC12 cells were treated with mock or 1 μM midi-GAGR, dextran, or alginate, and stained with antibodies to α-tubulin (red) and pCREB (green) along with DAPI (blue).

FIG. 9B: Midi-GAGR increases the levels of phosphorylated CREB in the nucleus.

Depicted is a bar graph showing the average intensities of pCREB in the nucleus and cytoplasm (n=100 cells, Mean±SEM).

FIGS. 10A-10D: Midi-GAGR increases the levels of phosphorylated CREB in the nucleus. Mouse embryonic cortical neurons were treated with mock or 1 μM dextran (FIG. 10A), alginate, high acyl gellan gum, or midi-GAGR (FIG. 10B) and processed for immunocytochemistry using antibodies to α-tubulin (red) and pCREB (green) along with DAPI (blue).

FIGS. 10C-10D: Midi-GAGR increases the levels of phosphorylated CREB in the nucleus. Depicted are bar graphs showing the average intensities of pCREB in the nuclei.

FIGS. 11A-11D: Midi-GAGR has a neuritogenic activity. Rat embryonic cortical neurons were treated with mock (FIG. 11A) or 1 μM midi-GAGR (FIG. 11B) for 2 days and then with none (FIGS. 11A-11B) or 25 μM 4-HNE (FIG. 11C: pretreated with mock; FIG. 11D: pretreated with midi-GAGR) for 1 day. Neurons were processed for immunocytochemistry using antibody to synaptophysin (green) and phalloidin (actin: red) (scale bars=75 μm).

FIGS. 12A-12C: Midi-GAGR has neuritogenic and synaptogenic activities. Depicted is a line graph showing the numbers of dendrite crossings (FIG. 12A), and a bar graph showing the number of synaptic clusters (per 15 μm) (FIG. 12B) in rat embryonic cortical neurons treated with mock, 1 μM dextran or 1 μM midi-GAGR. The numbers of dendritic crossing and synaptic clusters were quantified by Scholl ring analysis and Metamorph (n=30 neurons×2, Mean±SEM, *=p<0.001).

FIG. 12C: Midi-GAGR has a neuritogenic activity. Depicted is a line graph showing the numbers of crossings counted in neurons pretreated with mock, 1 μM dextran, 100 μM ascorbate, 1 μM high acyl gellan gum or 1 μM midi-GAGR, and then treated with 25 μM 4-HNE (n=30 neurons×2, Mean±SEM, * (dark colored star)=p<0.01, * (light colored star)=p<0.001 compared to mock).

FIG. 13A: Midi-GAGR has a neurite outgrowth-enhancing effect in the presence of 25 μM 4-HNE. Depicted is a bar graph showing the total neurite length (um) of mouse embryonic cortical neurons treated with mock or 1 nM, 10 nM, 100 nM, 1 μM or 10 μM midi-GAGR in the presence of 25 μM 4-HNE (mean±SEM, n=40 cells. One way ANOVA, Bonferroni post hoc test, **: p<0.05; ****: p<0.0001).

FIG. 13B: Midi-GAGR has a neurite outgrowth-enhancing effect in the presence of 200 μM H₂O₂. Depicted is a bar graph showing the total neurite length (μm) of cortical neurons treated with mock or 1 nM, 10 nM, 100 nM, 1 μM or 10 μM midi-GAGR in the presence of 200 μM H₂O₂ (mean±SEM, n=3 cells. One way ANOVA, Bonferroni post hoc test, *: p<0.05; ***: p<0.0001).

FIG. 14A-14F: Midi-GAGR enhances neuritogenesis and actin filopodia formation in differentiated neuro2A (N2A) cells. N2A cells were starved in serum-free medium plus mock (FIG. 14A) or 1 μM midi-GAGR (FIG. 14B) for 3 days and stained by antibody to α-tubulin (green) and phalloidin (actin: red). Actin filopodia were observed in the neurites of midi-GAGR-treated cells (FIG. 14D), but not in mock treated cells (FIG. 14C). After treatment with mock or midi-GAGR, cells were treated with mock (FIG. 14E) or 100 μM H₂O₂ (FIG. 14F) for one day and processed for immunocytochemistry as described above. (Scale bars in FIGS. 14A-14F=75 μm.)

FIG. 15A: Midi-GAGR-treated N2A cells form neurites. Depicted is a bar graph showing the numbers of neurites in N2A cells starved in serum-free medium plus mock or 1 μM midi-GAGR for 3 days (n=90, 30 cells×3 independent experiments, Mean±SEM, *=p<0.001).

FIG. 15B: Midi-GAGR treated N2A cells form neurites in the presence of 25 μM 4-HNE and 200 μM H₂O₂. Depicted is a bar graph showing the average total neurite lengths of N2A cells starved in serum-free medium plus mock or 1 μM midi-GAGR for 3 days and then treated with mock, 100 or 200 μM H₂O₂, or 25 or 50 μM 4-HNE (n=90, 30 cells×3 independent experiments, Mean±SEM, * (blue star)=p<0.001 compared to mock, * (red star)=p<0.001 compared to treatment with free radicals minus midi-GAGR).

FIG. 16: In vitro blood-brain barrier (BBB) filter system. Depicted is a schematic showing the generation of an in vitro BBB consisting of bEND3 cells and astrocytes.

FIG. 17: Midi-GAGR penetrates an in vitro blood-brain barrier (BBB). Depicted is a bar graph showing the average total neurite length of N2A cells starved for 3 days in serum-free medium without (no F) or under the in vitro BBB filter system (F) on which mock or 1 or 10 μM midi-GAGR was added and then treated with mock or 25 μM 4-HNE (n=60, 30 cells×2 independent experiments, Mean±SEM, *=p<0.001).

FIG. 18: Midi-GAGR binds to NCAM1. Depicted is a bar graph showing the normalized spectrum count for the binding of mock (control) or midi-GAGR to neurexin, carboxypeptide D, or NCAM1. Data is the result of two independent experiments. Midi-GAGR was chemically crosslinked to epoxy-activated sepharose resins that were used for affinity chromatography. Proteins that bound to midi-GAGR-affinity column were purified using the midi-GAGR affinity column and identified by mass spectrometry (MS/MS) protein sequencing.

FIG. 19A: The standard curve of emissions at 520 nm (relative fluorescence unit: RFU) of ANTS at 0, 0.1, 0.3, 1, 3, and 10 mM (n=5).

FIG. 19B: The standard curve of absorbances at 490 nm for 0, 7.4, and 74 nM, 0.74 and 7.4 mM of midi-GAGR (n=3).

FIG. 19C: Fluorescent and colorimetric values of ANTS-midi-GAGR before and after three 75% ethanol washes (Mean±Standard Error, n=5).

FIG. 20A: The RFUs (Mean±Standard Error) of the brain cytosols from animals administered with none or 1 mM ANTS-midi-GAGR. Brains were dissected out of rats that were sacrificed at 6 h after administration (n=3).

FIG. 20B: The RFUs (Mean±Standard Error) of the sera from animals administered with none or 1 mM ANTS-midi-GAGR. Serum samples were collected from rats that were sacrificed at 6 h after administration (n=4).

FIG. 20C: The RFUs (Mean±Standard Error) of the supernatants and pellets of TCA precipitation and ethanol precipitation of sera (n=5).

FIG. 21: Schematic illustration showing a method to examine the BBB-permeability and, possibly, tissue distribution of a target polysaccharide in animal. A polysaccharide was used with fluorescent 8-aminonaphthalene-1,3,6-trisulfonic acid disodium salt (ANTS) for tracking in animal. ANTS-tagged polysaccharide was separated from unconjugated free ANTS using 75% ethanol. After ANTS-polysaccharide was intra-nasally administered into animals, the amounts of ANTS-polysaccharide in the brain and the serum were quantified by fluorocytometry. Free ANTS-polysaccharide was separated from serum proteins using 75% ethanol and trichloroacetic acid (TCA).

FIGS. 22A-22D: Images of the HRP-stained cell bodies of infraorbital nerve (ION) of rats: Uncut positive control (con) (FIG. 22A), nanotube-alone injection (FIG. 22B), or nanotube+midi-GAGR (Midi) injection (FIG. 22C). FIG. 22D displays bar graphs showing the numbers of ganglionic neuron cell bodies positive.

FIG. 23: Bar graphs show the total number of cells positive for HRP in trigeminal ganglia from uncut control rats, post-transection nanotube-treated rats, and post-transection midi-GAGR-encapsulated nanotube-treated rats. Data represent mean±SEM (n=3 rats/group, * p<0.05). Rats were applied with nanotubes or midi-GAGR-encapsulated nanotubes after infraorbital nerve transection. After four weeks, rat whisker follicles were labeled with HRP, followed by dissection of trigeminal ganglia. The number of cell bodies labeled with HRP in trigeminal ganglion sections was counted using a light microscope.

FIG. 24: The number of HRP labeled cells in midi-GAGR-treated animals did not differ from un-cut control animals after 8 weeks. Rats were applied with nanotubes or midi-GAGR-encapsulated nanotubes after infraorbital nerve transection. After eight weeks, rat whisker follicles were labeled with HRP, followed by dissection of trigeminal ganglia. The number of cell bodies labeled with HRP in trigeminal ganglion sections was counted using a light microscope. FIG. 24 displays a bar graph showing the total number of cells positive for HRP in trigeminal ganglia from uncut control rats, post-transection nanotube treated rats, and post-transection midi-GAGR-encapsulated nanotube treated rats. Data represent mean±SEM, * p<0.05.

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

### DEFINITIONS

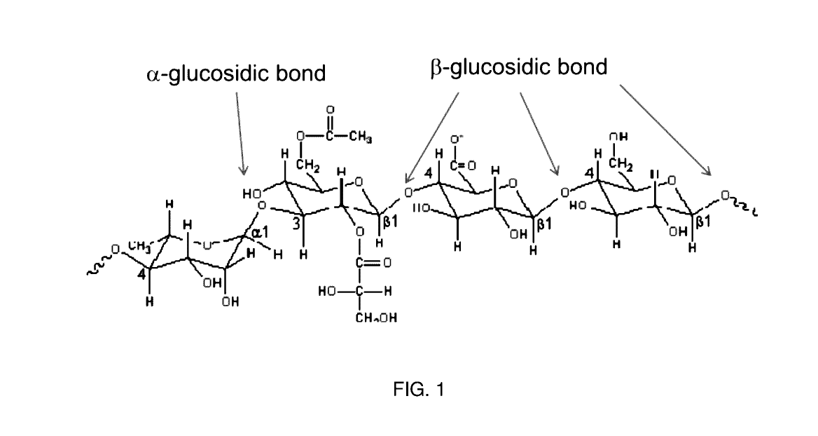

As used herein, the term “LA-GAGR” refers to low acyl gellan gum. LA-GAGR is a polysaccharide based on a tetrasaccharide repeating unit consisting of glucose derivatives such as glucuronic acid, mannose, and rhamnose connected by (1-3)-α and (1-4)-β, and has an average molecular weight of 99,600 g/mole (LA-GAGR: [D-Glc(β1→4)D-GlcA(β1→4)D-Glc(β1→4)L-Rha(α1→3]n) (FIG. 1). The molecular weights of the repeating units are reported to be 700-1,000 g/mole, depending on their functional groups and branches. Gellan gum is produced by fermentation of calcium hydrates in Sphingomonas elodea monocultures extracted from natural resources. Primarily used as a food additive, LA-GAGR may be found in food products such as baked goods, cake icings, various sweets, jellies and spreads, jams, puddings, sauces, dairy products, and microwave-ready foods. LA-GAGR can also be used in cosmetic and hygiene products such as makeup, facial masks, creams and lotions. In pharmaceuticals, LA-GAGR is used to make tablets that are easy to swallow, as well as to adjust the rate of release of medicinal compounds in the body.

As used herein, the term “neurodegenerative disorders” refers to any central nervous system (CNS) or peripheral nervous system (PNS) disease that is associated with neuronal or glial cell defects including but not limited to neuronal loss, neuronal degeneration, neuronal demyelination, gliosis (i.e., astrogliosis), or neuronal or extra-neuronal accumulation of aberrant proteins or toxins (e.g., β-amyloid, or α-synuciein). The neurological disorder can be chronic or acute. Non-limiting examples of various chronic and acute neurological diseases include Parkinson's disease, Alzheimer's disease, amyotrophic lateral sclerosis, myasthenia gravis, multiple sclerosis, microbial infections, stroke, Pick's disease, dementia with Lewy bodies, Huntington disease, chromosome 13 dementias, Down's syndrome, cerebrovascular disease, Rasmussen's encephalitis, viral meningitis, NPSLE, amyotrophic lateral sclerosis, Creutzfeldt-Jacob disease, Gerstrnann-Straussler-Scheinker disease, transmissible spongiform encephalopathies, ischemic reperfusion damage (e.g. stroke), brain trauma, spinal cord injury, microbial infection, chronic fatigue syndrome, Mild Cognitive Impairment, movement disorders (including ataxia, cerebral palsy, choreoathetosis, dystonia, Tourette's syndrome, kernicterus), tremor disorders, leukodystrophies (including adrenoleukodystrophy, metachromatic leukodystrophy, Canavan disease, Alexander disease, Pelizaeus-Merzbacher disease); neuronal ceroid lipofucsinoses, ataxia telangectasia, and Rett Syndrome.

As used herein, the term “neuroprotective” refers to the relative preservation of neuronal structure and/or function. In a case of ongoing neurodegenerative insult, the relative preservation of neuronal integrity implies a reduction in the rate of neuronal loss over time. Neuroprotection aims to prevent or slow disease or injury progression and secondary injuries by halting or at least slowing the loss of neurons.

As used herein, the term “effective amount” refers to an amount of an agent that is sufficient to exert a therapeutically significant neuroprotective effect, a therapeutically significant neurotrophic effect in a subject diagnosed with a neurodegenerative disorder. The therapeutically effective amounts to be administered will depend on the severity of the condition and individual subject parameters including age, physical condition, size, weight, and concurrent treatment. In certain embodiments, a maximum dose can be used, that is, the highest safe dose according to sound medical judgment. However, a lower dose or tolerable dose may be administered for medical reasons, psychological reasons or for virtually any other reason.

The actual dosage amount of a composition administered to the subject, such as a human subject, can be determined by physical and physiological factors such as body weight, severity of condition, the type of disease being treated, previous or concurrent therapeutic interventions, idiopathy of the subject and on the route of administration. The practitioner responsible for administration will, in any event, determine the concentration of active ingredient(s) in a composition and appropriate dose(s) for the individual subject.

As used herein, the term “therapeutic agent” refers to any biologic agent, molecule, compound, and/or substance that is used for the purpose of treating and/or managing a disease or disorder. As used herein, “agent” refers to any biologic agent, molecule, compound, methodology and/or substance for use in the prevention, treatment, management and/or diagnosis of a disease or disorder. As used herein, the terms “prevent,” “preventing”, and “prevention” in the context of the administration of a therapy to a subject refer to the prevention or inhibition of the recurrence, onset, and/or development of a disease or condition, or a symptom thereof, in a subject resulting from the administration of a therapy (e.g., a prophylactic or therapeutic agent), or a combination of therapies (e.g., a combination of prophylactic or therapeutic agents).

As used herein, the terms “treat,” “treatment,” and “treating” in the context of the administration of a therapeutic agent to a subject refer to the reduction or inhibition of the progression and/or duration of a disease or condition, the reduction or amelioration of the severity of a disease or condition, and/or the amelioration of one or more symptoms thereof resulting from the administration of one or more therapies.

The terms “subject,” “individual”, and “patient” are defined herein to include animals such as mammals, including but not limited to, primates, cows, sheep, goats, horses, dogs, cats, rabbits, guinea pigs, rats, mice or other bovine, ovine, equine, canine, feline, rodent, or murine species. In one embodiment, the subject is a mammal (e.g., a human) afflicted with a neurodegenerative disorder.

Pharmaceutical compositions are characterized as being sterile and pyrogen-free. As used herein, “pharmaceutical compositions” include compositions for human and veterinary use. Methods for preparing pharmaceutical compositions of the invention are within the skill in the art, for example, as described in Remington's Pharmaceutical Science, 17th ed., Mack Publishing Company, Easton, Pa. (1985), the entire disclosure of which is herein incorporated by reference.

Pharmaceutical compositions can also comprise conventional pharmaceutical excipients and/or additives. Suitable pharmaceutical excipients include stabilizers, antioxidants, osmolality-adjusting agents, buffers, and pH-adjusting agents. Suitable additives include physiologically biocompatible buffers (e.g., tromethamine hydrochloride), chelants (such as, e.g., DTPA or DTPA-bisamide) or calcium chelate complexes (e.g., calcium DTPA, CaNaDTPA-bisamide), or, optionally, additions of calcium or sodium salts (e.g., calcium chloride, calcium ascorbate, calcium gluconate or calcium lactate). Pharmaceutically-acceptable carriers can include water, buffered water, normal saline, 0.4% saline, 0.3% glycine, hyaluronic acid and the like. Pharmaceutical compositions can be packaged for use in liquid form, or can be lyophilized.

For solid pharmaceutical compositions, nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can comprise any of the carriers and excipients listed above and 10-95%, preferably 25%-75%, of the neuroprotective agent. A pharmaceutical composition for aerosol (inhalational) administration can comprise 0.01-20% by weight, preferably 1%-10% by weight, of neuroprotective agent encapsulated in a liposome, as described above, and a propellant. A carrier can also be included as desired, e.g., lecithin, for intranasal delivery.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. In other non-limiting examples, a dose may also comprise from at least about 1 microgram/kg/body weight, including about 5, about 10, about 50, about 100, about 200, about 350 weight, about 500 microgram/kg/body weight; or at least about 1 milligram (mg)/kg/body weight, such as about 5, about 10, about 50, about 100, about 200, about 350, about 500, to about 1000 mg/kg/body weight or more per administration, and any range derivable therein.

General Description

Described herein are methods for generating midi- and mini-GAGR, compositions comprising midi- and mini-GAGR, and applications and methods employing such compositions.

Described herein are methods of generating digestion products of low acyl gellan gum (LA-GAGR). LA-GAGR is enzymatically digested by α(1→3) glucosidase. Allowing the digestion to proceed for 24, 48, or 72 hours produces three different digestion products: mega-GAGR, midi-GAGR, and mini-GAGR, respectively. Compositions described herein comprise midi-GAGR and/or mini-GAGR. In accordance with the present disclosure, the compositions described herein have been found to be neuroprotective. In certain specific embodiments, the compositions described herein can be used according to methods described herein to treat neurodegenerative disorders, e.g. Alzheimer's disease, Parkinson's disease, multiple sclerosis, amyotrophic lateral sclerosis, spinal cord injury, brain trauma, and peripheral nerve injury.

In yet other embodiments described herein are methods for treating degenerative neurological disorder using the compositions described herein.

While not wishing to be bound by any particular theory, the neuroprotective effect of midi-GAGR is now believed to be attributable to its antioxidant activity, and its ability to bind and interact with neural cell adhesion molecule 180 (NCAM-180) and neurofascin-186. Because of midi-GAGR's antioxidant activity and ability to bind and interact with NCAM-180, compositions described herein comprising midi-GAGR can be used according to the methods described herein for the treatment of degenerative neurological disorders.

LA-GAGR is already used as a food additive, and is approved by the FDA. It has few side effects in humans, even at high doses (˜200 mg/kg). LA-GAGR has good oral bioavailability and is relatively stable in serum, but biodegradable and cost effective (˜$67.00 per 500 g). Midi- and mini-GAGR are expected to have as few side effects in humans as LA-GAGR does.

LA-GAGR may be enzymatically digested by α(1→3) glucosidase to generate several digestion products. A 24 h enzymatic digestion of LA-GAGR results in a digestion product having an average molecular weight of approximately 30,245 g/mole. “Midi-GAGR” is generated after 48 h enzymatic digestion of LA-GAGR. Midi-GAGR has a molecular weight of approximately 4,774 g/mole. “Mini-GAGR” is generated after 72 h enzymatic digestion of LA-GAGR. Mini-GAGR has a molecular weight of approximately 718 g/mole. This is close to the molecular weight of the basic repeating sugar units of LA-GAGR. LA-GAGR may be similarly digested by other glucosidases, for example, α(1→4) glucosidase.

In one embodiment, pharmaceutical compositions disclosed herein comprise a neuroprotective agent, wherein the agent is a digestion product of LA-GAGR, mixed with a pharmaceutically acceptable carrier. In one particular embodiment, the agent is the digestion product midi-GAGR, mini-GAGR or a combination thereof.

PNT for Slow Release of Drugs at Peripheral Nerve Injury

Given that midi-GAGR increases the proteins involved in neuritogenesis in the brain, without wishing to be bound by theory, it is believed that midi-GAGR and mini-GAGR have a similar neurogenic effect on the peripheral nervous system.

Peptide nanotube (PNT) can be used for the controlled and slow release of drugs (e.g., a neurogenic midi-GAGR) from the injection site to the surrounding areas. Self-assembling PNTs have excellent biocompatibility and mechanical, chemical, and thermal stability. PNTs can withstand highly acidic (pH=1) and highly basic (pH=14) solutions. In vitro dialysis studies examining entrapped drug release profile of PNTs have revealed that drug release from PNTs can be accelerated when pH of the solution is either increased to 10.0-11.0 or decreased to 2.0-3.0, but PNTs can deliver sustained release at physiological pH.

PNT can be used to slowly release the neurogenic drug, midi-GAGR, to the region that surrounds severed peripheral nerves and thus helps the regeneration of axon. To generate an animal model of peripheral nerve cut, the examples herein describe the use of a left infraorbital nerve transaction to cut the middle of axons that originate from the cell bodies of the trigeminal ganglia under the eyes and innervate into the sensory nerve endings linked to the whiskers of rat. Midi-GAGR encapsulated in peptide nanotube (PNT) is injected into the site of peripheral nerve cut. If midi-GAGR exerts its neurogenic effect, the axons regenerate from the cell bodies of the trigeminal ganglia and re-innervate into the whiskers. Horseradish peroxidase (HRP, retrograde marker) is injected to the nerve endings underneath the whiskers and, if axons regenerate, is retro-transported back to the cell bodies of the trigeminal ganglia. PNT prevents the dispersion and dilution of locally injected drug from the injection site.

Trigeminal nerve is the fifth and largest cranial nerve that divides mainly into three branches—1) Ophthalmic nerve, 2) Maxillary nerve, and 3) Mandibular nerve. Branches of these nerves supply to most of the face including nose, mouth and eyes. Ophthalmic and maxillary nerve are purely sensory, whereas mandibular nerve contains both sensory and motor fibers. Fibers of trigeminal nerve have their cell bodies in trigeminal ganglion. One of the three branches of trigeminal nerve, maxillary nerve, is called infraorbital nerve when it enters infraorbital canal. Infraorbital nerve mainly supplies to lower eyelid, parts of nasal vestibule and upper lip in human. But in rodents, infraorbital branch of maxillary nerve innervates mystacial vibrissae.

Infraorbital nerve injury in neonatal rats has been studied to determine the degree of plasticity in developing somatosensory nervous system. Several studies have shown that infraorbital nerve lesion causes perturbation in somatotopic pattering of barrel cortex during postnatal day 0 to 4. In the later stages of development, peripheral nerve lesion does not affect barrel cortex pattern. In addition, neonatal infraorbital transection causes death of neurons in transgeminal ganglion and ventrobasal thalamic complex. Cell death in trigeminal ganglion following infraorbital nerve transection in neonates ranges from 19% to 38%. This would cause the degeneration of central processes which may explain the perturbations observed in barrel cortex following peripheral injury. Little attention has been given to the responses following peripheral nerve injury in adult animals. The degeneration and regeneration of infraorbital nerve in adult rats has been demonstrated at different time points after nerve transection or nerve crush. Large diameter myelinated axon terminals innervating vibrissae started undergoing degeneration as early as 24 h post lesion. In one week following nerve lesion, no intact axon or neural element were seen in vibrissae. In post-transection animals, there were no signs of intact or regenerating nerve after 2 weeks but vibrissae sections from post-crush animals showed numerous unmyelinated small-diameter axons entering the vibrissae at 2 weeks and complete innervation in 1.5 months. Electron microscopic examination of vibrissae 1.5 months after ION complete transection showed scant nerve supply with no signs of neural elements. Animals that underwent complete ION transection showed several myelinated and unmyelinated axonal innervation in vibrissae after 3 months, but disorganized nerve network did not resemble highly ordered nerve innervation in vibrissae from normal rats. Comparison of regeneration study in post-crush and post-transection animals explains the importance of conduit guidance in nerve regeneration.

Peripheral axonal transection and regeneration models have proven to be powerful tools to study cellular response to nerve injury, mechanisms of regeneration, and processes leading to neurodegeneration. In addition, an axotomy model can be used to test therapeutic agents for their neuro-regenerative or neurotrophic property. Peripheral axonal transection can be made with great accuracy and axonal fiber regeneration can be measured and compared without high variability. Here, adult rat infraorbital nerve transection was used as a model to examine the neuro-regenerative properties of midi-GAGR. Left infraorbital nerve was completely transected at the site of its exit from infraorbital foramen and then peptide nanotubes-encapsulated with midi-GAGR was applied at the site of injury to examine its effect on proximal axonal stump regrowth using histochemistry. Retrograde tracer (horseradish peroxidase) injection into whisker follicle was used to label regenerated axonal fiber cell bodies in trigeminal ganglia. Given that infraorbital nerve did not innervate the vibrissae pad as long as 1.5 months post-transection in previous studies, labeling was performed 4 and 8 weeks post-transection. Midi-GAGR enhances neuro-regeneration after infraorbital nerve transection in rats.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention and without departing from the spirit and scope thereof, can make various changes and modifications of the invention to adapt it to various usages and conditions.

### Example 1

### Generation of Midi-GAGR and Mini-GAGR

Low acyl gellan gum (LA-GAGR) has an average molecular weight of 99,600 g/mole, based on a tetrasaccharide repeating unit consisting of glucose and glucose derivatives such as glucuronic acid, mannose, and rhamnose connected by (1-3)-α and (1-4)-β (LA-GAGR: [D-Glc(β1→4)D-GlcA(β1→4)D-Glc(β1→4)L-Rha(α1→3)]ₙ). Depending on their functional groups and branches, the molecular weights of the repeating units are 700-1,000 g/mole.

LA-GAGR was enzymatically digested by enzymatic hydrolysis using α(1→3) glucosidase (Sigma Aldrich, St. Louis, Mo.), generating midi-GAGR and mini-GAGR. The digestion was performed in 0.1 M acetate buffer (pH 5) containing 1% salicin (cofactor) in glass tubes. A 1% salicin solution was prepared by dissolving 1 g salicin in 100 ml of 0.1 M acetate buffer at pH 5. The salicin solution was then incubated for 6-8 minutes before use. The mixture was shaken lightly in an incubator at 37° C. and 80 rpm. At 24, 48, and 72 h after the start of enzymatic hydrolysis, the glass tubes were removed from the incubator, placed in a hot water bath for 5 minutes, and then moved to an ice bath to stop the digestion reaction. The 24, 48, and 72 hour digestion products were named mega-GAGR, midi-GAGR, and mini-GAGR, respectively. The samples were refrigerated until molecular weight measurement.

Prior to molecular weight measurement, samples were placed in a vacuum dryer at −60 cmHg gauge at 70° C. The tubes were removed from the vacuum dryer after approximately 8 ml of 0.1 M acetate buffer had evaporated. 1 g of LA-GAGR digestion sample was re-suspended in 30 ml of distilled water. Molecular weights of the resulting LA-GAGR fragments were then measured using a Parallel Plate Rheometer (PPR, Rheometrics, Inc.) equipped with rheometer software (TA Orchestrator, TA Instrument, Inc.). Molecular weights of the LA-GAGR digestion products were determined on the basis of the viscosity-storage modulus profiles of the samples using the RheoAnalyzer program developed by TomCoat Oy, Inc., Finland. The validity of the RheoAnalyzer program was verified by running polystyrene standard (NBS 706) on the PPR and determining the molecular weight of NBS 706 from its viscosity profile. The results of the molecular weight measurements are show in Table 1.

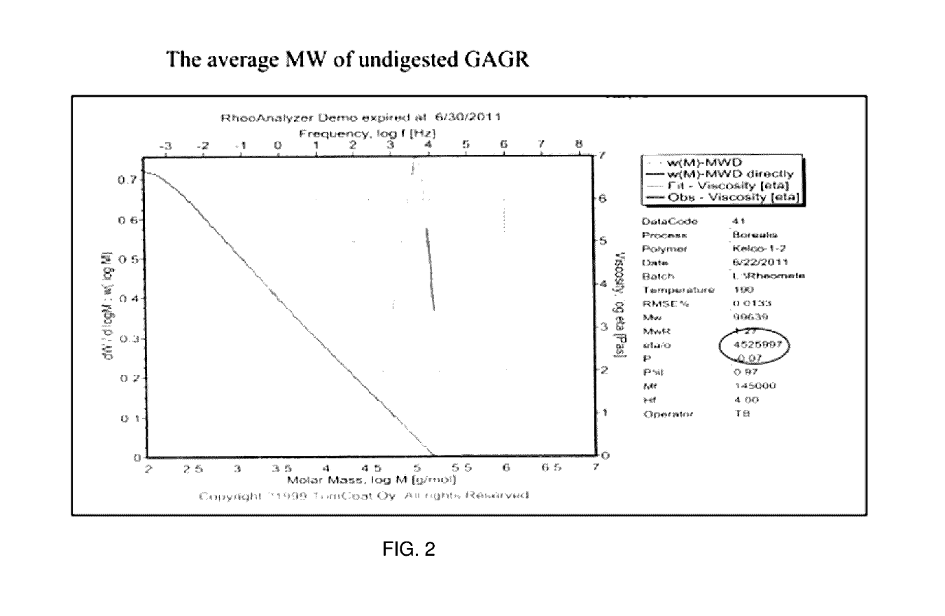

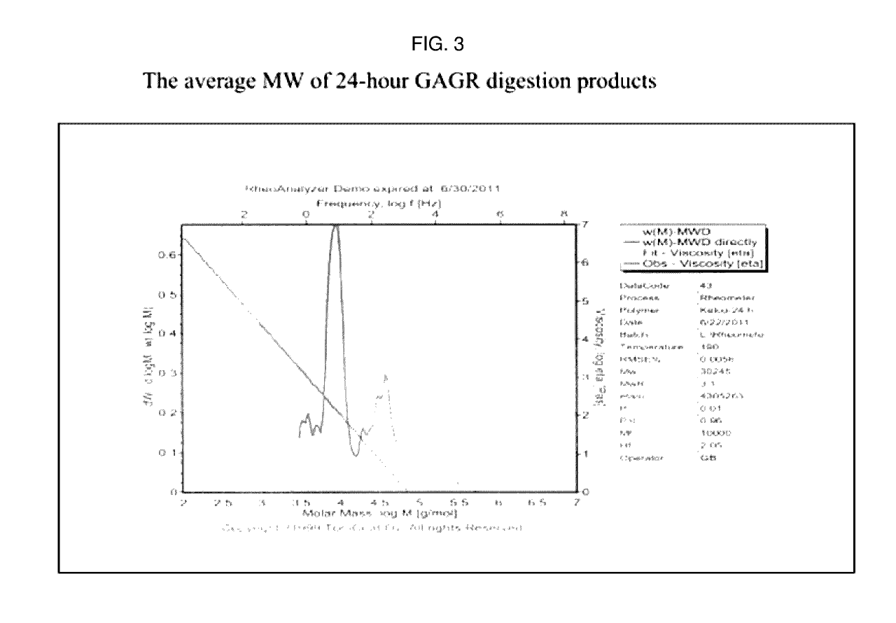

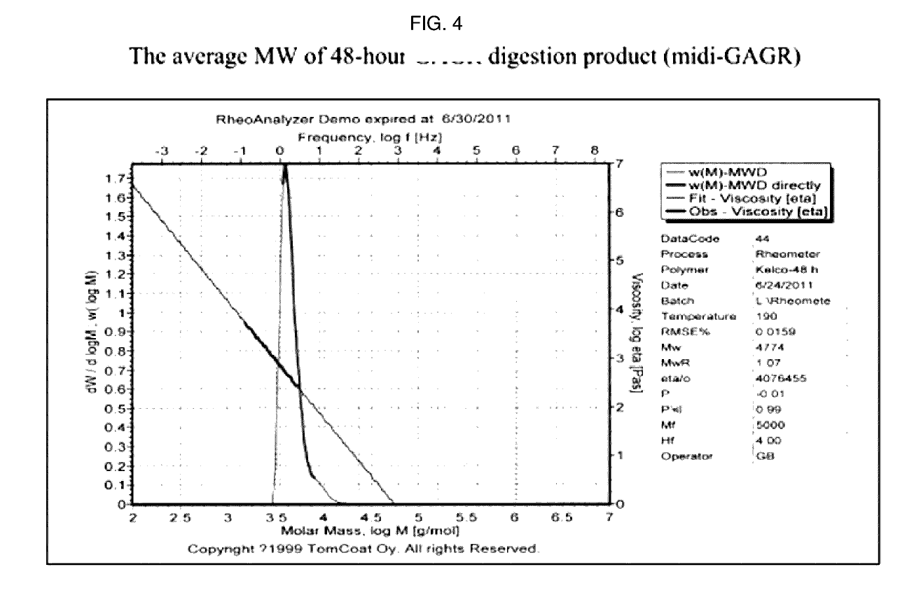

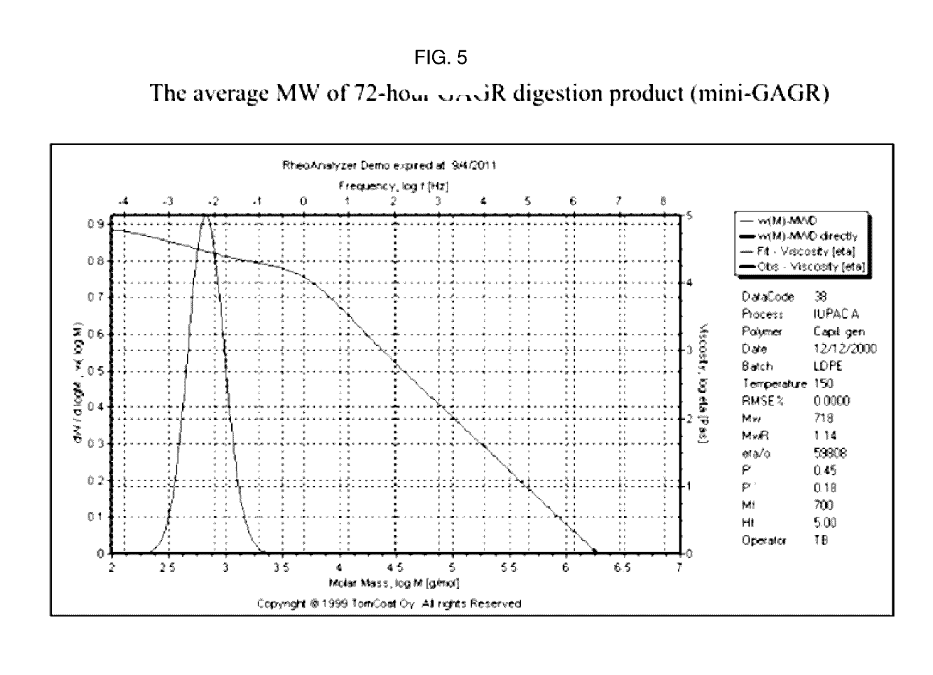

FIG. 2 shows the readout of the rheometer measurement for LA-GAGR. The undigested LA-GAGR had an average molecular weight of approximately 99,636 g/mole, which is close to the reported average of 99,600 g/mole. After 24 hours of enzymatic digestion, the average molecular weight of the LA-GAGR digestion product was reduced to approximately 30,245 g/mole (FIG. 3). The three peaks shown in FIG. 3 indicate that α(1→3) glucosidase breaks down LA-GAGR in multiple steps. The 48-hour digestion of LA-GAGR, midi-GAGR, yielded a product of approximately 4,775 g/mole (FIG. 4). The single narrow peak shown in FIG. 4 indicates that 48-hour enzymatic digestion yielded a polysaccharide product of an approximate equivalent length (molecular weight). 72-hour enzymatic digestion of LA-GAGR yielded a product of approximately 718 g/mole (mini-GAGR), which is close to the molecular weight of the basic repeating sugar units of LA-GAGR (FIG. 5).

TABLE 1
The molecular weights of LA-GAGR enzymatic digestions.

| | GAGR | | 48-hour | 72-hour |
|---|---|---|---|---|
| | Before | 24-hour | Enzymatic | Enzymatic |
| | Enzymatic | Enzymatic | Digestion | Digestion |
| | Digestion: | Digestion: | midi-GAGR | mini-GAGR |
| Molecular | 99,639 | 30,245 | 4,774 | 718 |
| Weight | | | | |
| (g/mole) | | | | |

### Example 2

### Mini-GAGR as a Neurotrophic Agent

Midi-GAGR protects cortical neurons from the neurocytotoxicity of amyloid β peptide.

Primary cultures of mouse embryonic cortical neurons (embryonic days of 17 [E17], 14 days in vitro [DIV14]) were treated with mock (H₂O) or 1 μM of dextran, alginate, high acyl gellan gum (HA-GAGR) or midi-GAGR for 6 h and then with 10 μM Aβ₄₂ for 48 h.

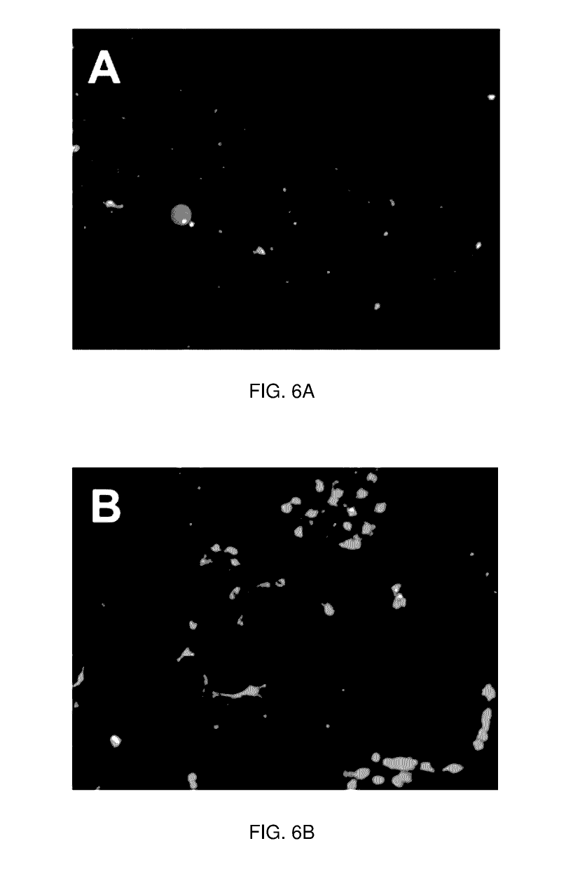

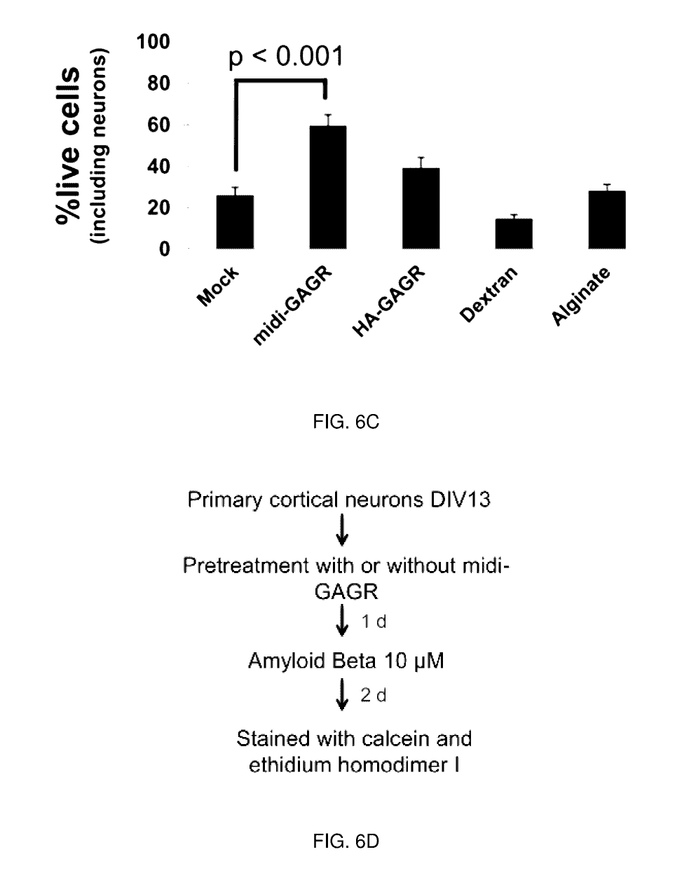

The viability of neurons was assed using LIVE/DEAD Viability/Cytotoxicity Assay Kit (Invitrogen Inc.). Midi-GAGR increased the percent of cell survival in neurons exposed to 10 μM Aβ₄₂ by ˜2.2 fold (FIGS. 6A-6C). The other sugar polymers had no effect.

Midi-GAGR is a strong antioxidant.

The antioxidant capacity of midi-GAGR was measured using ABTS Antioxidant Assay Kit (Zenbio Co.). Midi-GAGR showed strong anti-oxidant capacities (10 μM midi-GAGR=16.9 μM trolox). This was slightly stronger than antioxidant capacity of LA-GAGR (10 μM LA-GAGR=15.4 μM Trolox) (Table 2)

TABLE 2
Antioxidant Capacity of LA-GAGR and midi-GAGR

| ABTS assay | μM Trolox |
|---|---|
| LA-GAGR | 15.4 |
| midi-GAGR | 16.9 |

Midi-GAGR has neuroprotective effect on differentiated PC12 cells and primary neurons.

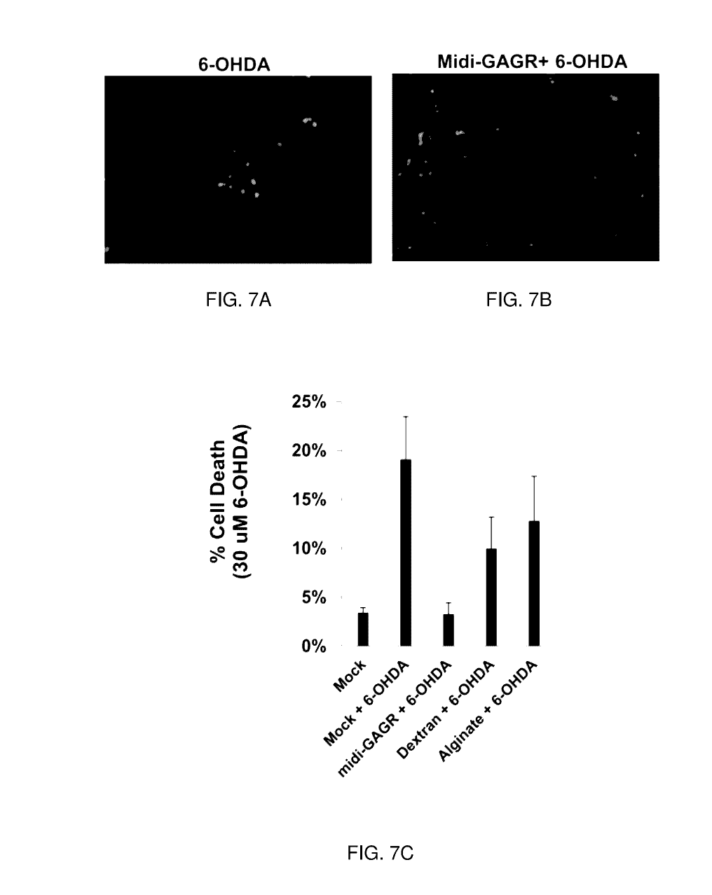

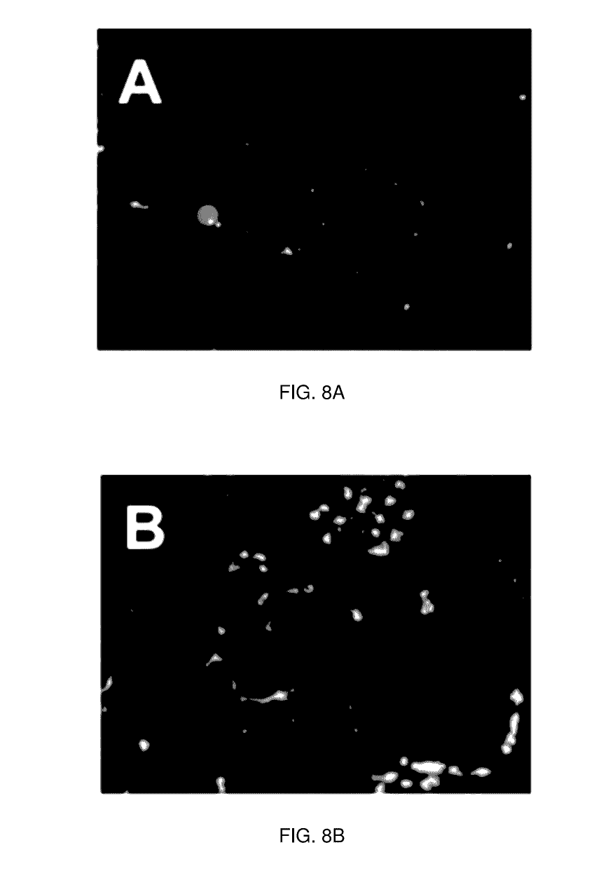

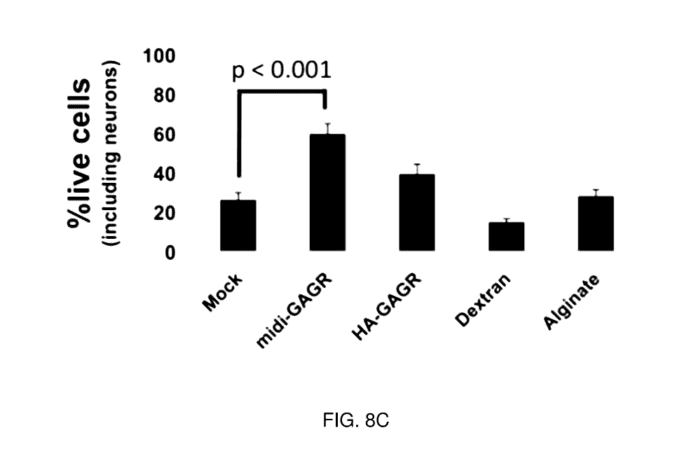

Differentiated dopaminergic PC12 cells and primary mouse embryonic cortical neurons (E17, DIV14) were treated with mock (H₂O) or 1 μM of dextran, alginate, HA-GAGR or midi-GAGR for 6 h and insulated by oxidative stressors (30 μM 6-OHDA [PC12] and 10 μM amyloid β peptide [cortical neurons]) for 24 h. The viability of neurons was assessed using LIVE/DEAD Viability/Cytotoxicity Assay Kit (Invitrogen). Midi-GAGR prevented 6-OHDA-induced cell death in differentiated PC12 cells while mock, dextran and alginated did not (FIGS. 7A-7C). Midi-GAGR also increased the percent of live neurons exposed to amyloid β peptide by ˜2.2 fold while other sugars did not (FIGS. 8A-8C). This shows that midi-GAGR has neuroprotective effect on both dopaminergic PC12 cells and primary neurons.

Treatment with midi-GAGR increased the levels of phosphorylated CREB in the nuclei of cortical neurons.

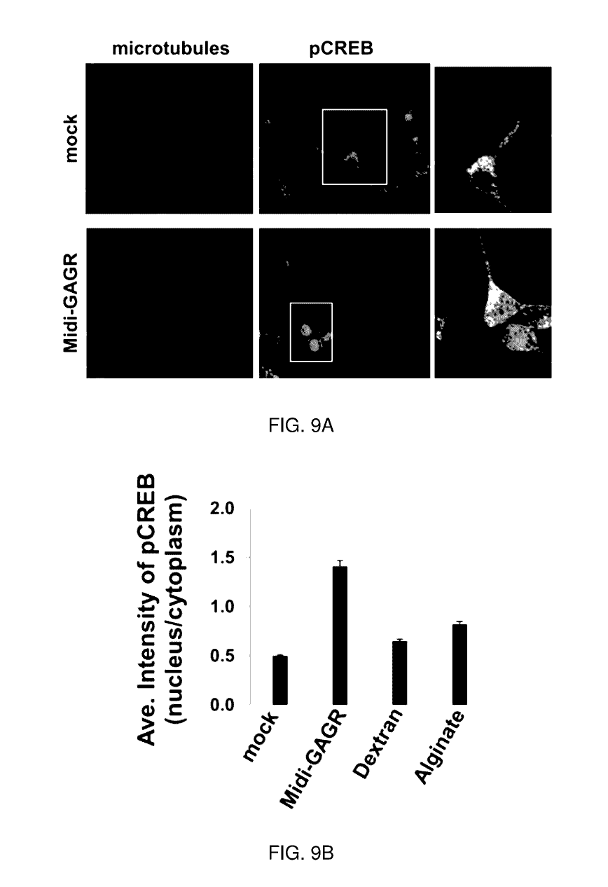

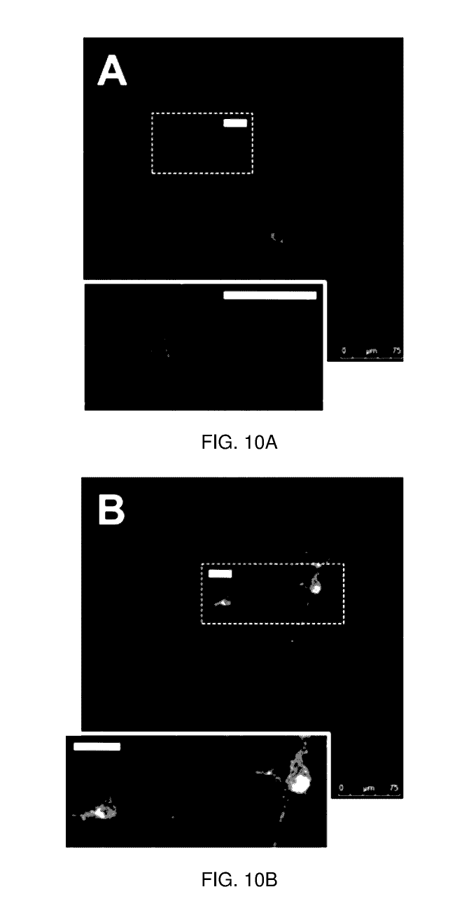

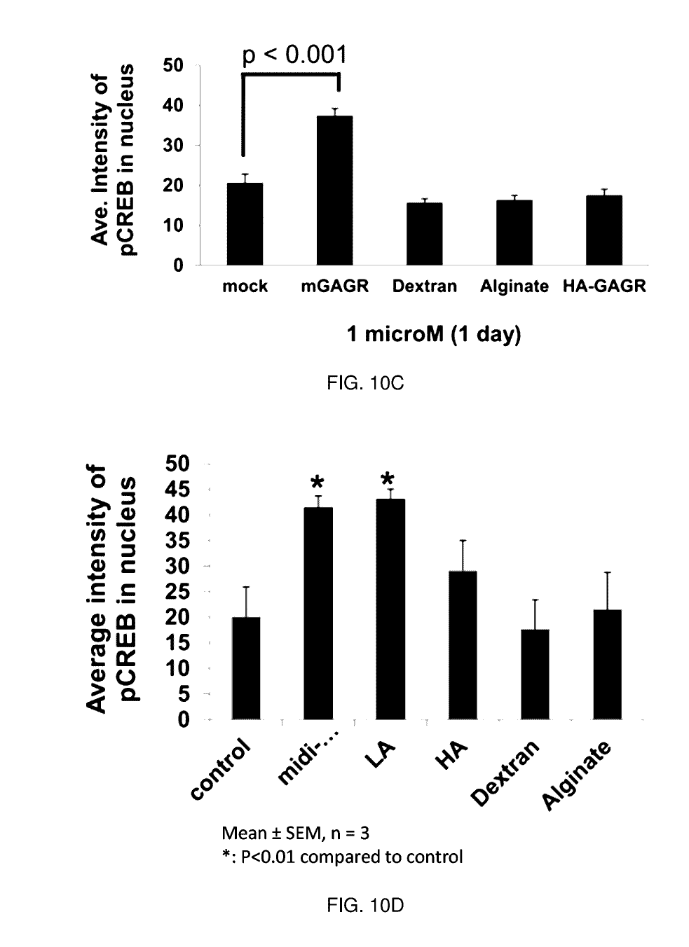

Differentiated PC12 cells and mouse embryonic cortical neurons (E17, DIV14) were treated with mock, dextran, alginate, HA-GAGR or midi-GAGR (1 μM) for 24 h and fixed in 3.7% paraformaldehyde. The fixed cells and neurons were immunostained with antibodies against α-tubulin and phospho-CREB (p-CREB) along with DAPI (nucleus). 1 μM midi-GAGR increased the levels of nuclear p-CREB by ˜3 fold in PC12 cells (FIGS. 9A-9B) and by ˜1.7 fold in cortical neurons (FIG. 10A-10C), while the nuclear levels of nuclear p-CREB were not significantly increased by mock, dextran, alginate or HA-GAGR. These data show that midi-GAGR activates a neurotrophic signaling pathway.

Midi-GAGR has a neuritogenic activity.

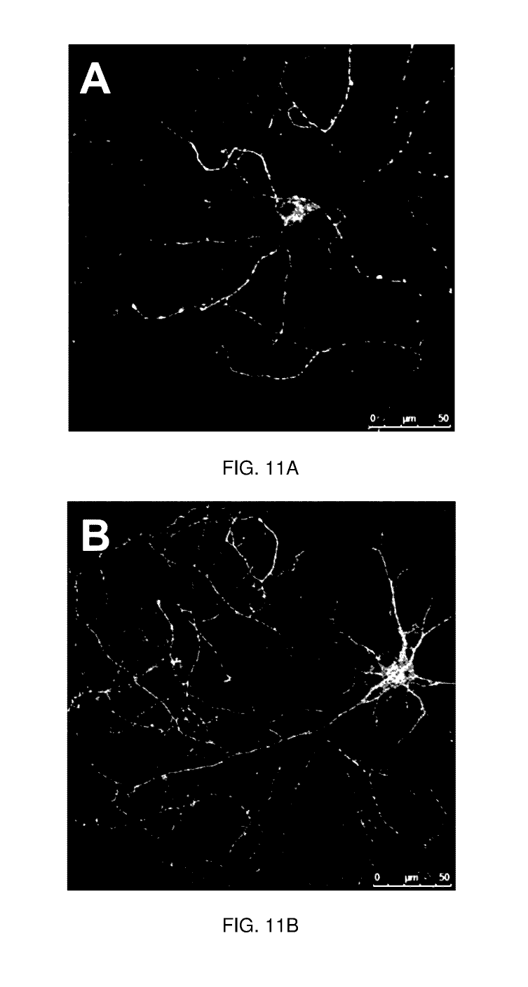

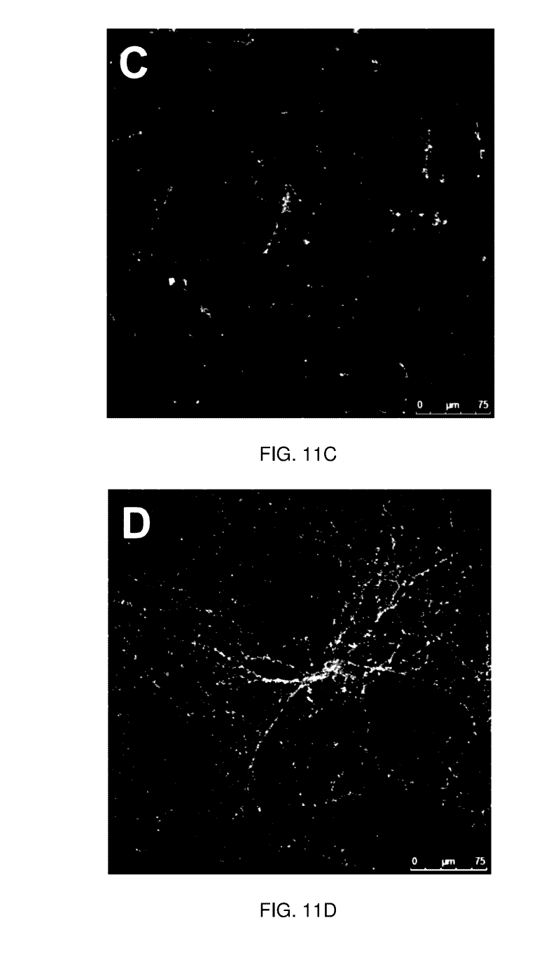

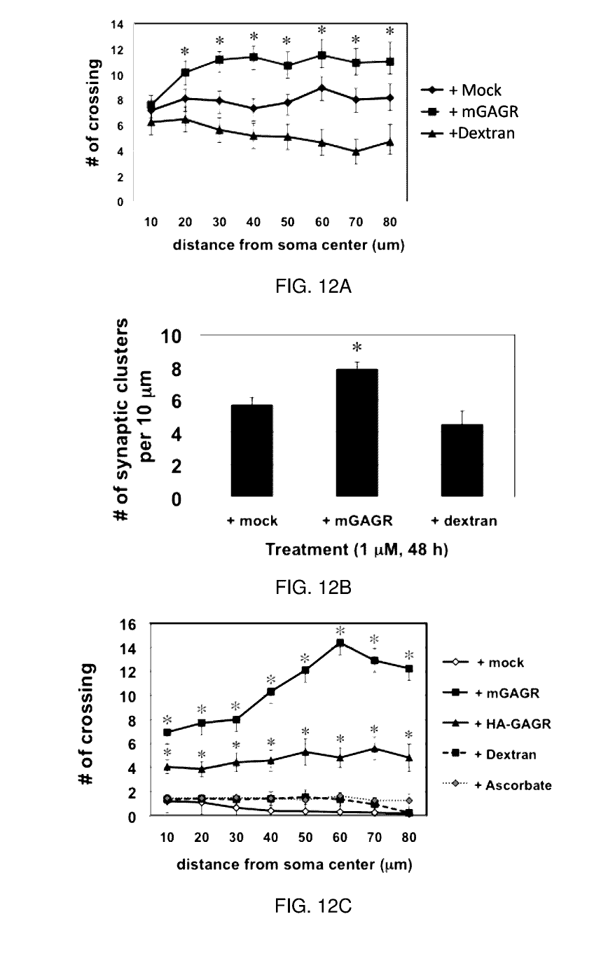

E16 rat embryonic cortical neurons (DIV14) were treated for 2 days with mock (FIG. 11A), 1 μM dextran or 1 μM midi-GAGR (FIG. 11B), fixed and immunostained with anti-synaptophysin antibody (green) and rhodamine-labeled phalloidin (actin: red). Scholl ring analysis was performed to quantify dendritic arbors the number of crossings as a function of distance from the soma between 0 and 120 μm (mean±S.E.M.). The average number of synaptic clusters per 10 μM of distal dendrite was also quantified. The number of crossings (0-120 μm) was higher in neurons treated with 1 μM midi-GAGR than mock- and dextran-treated neurons (FIG. 12A). The density of synaptic clusters was increased by ˜20% (p<0.03) in neurons treated with 1 μM midi-GAGR (FIG. 12B).

Neurons treated with midi-GAGR maintain their neurites in the presence of 25 μM 4-HNE.

Rat embryonic cortical neurons were treated with mock (FIG. 11C), 1 μM HA-GAGR (high acyl gellan gum), 1 μM dextran, 100 μM ascorbate or 1 μM midi-GAGR (FIG. 11D) for 2 days and then with 25 μM 4-hydroxynonenal (4-HNE), a lipid peroxide, for 1 day. After fixation, neurons were immunostained with anti-synaptophysin antibody (green) and rhodamine-phalloidin (actin: red). Scholl ring analysis was performed. Neurons pre-treated with mock, dextran or ascorbate lost neuritis (neuritic atrophy) in the presence of 4-HNE. Conversely, neurons pretreated with 1 μM HA-GAGR prior to 25 μM 4-HNE formed neuritis, but less branched. 25 μM 4-HNE did not cause severe neuritic atrophy in neurons pre-treated with 1 μM midi-GAGR (FIG. 12C). This result indicates that midi-GAGR prevents neuritic atrophy caused by 4-HNE.

Midi-GAGR has a neurite outgrowth-enhancing effect in the presence of 25 μM 4-HNE and 200 μM H₂O₂.

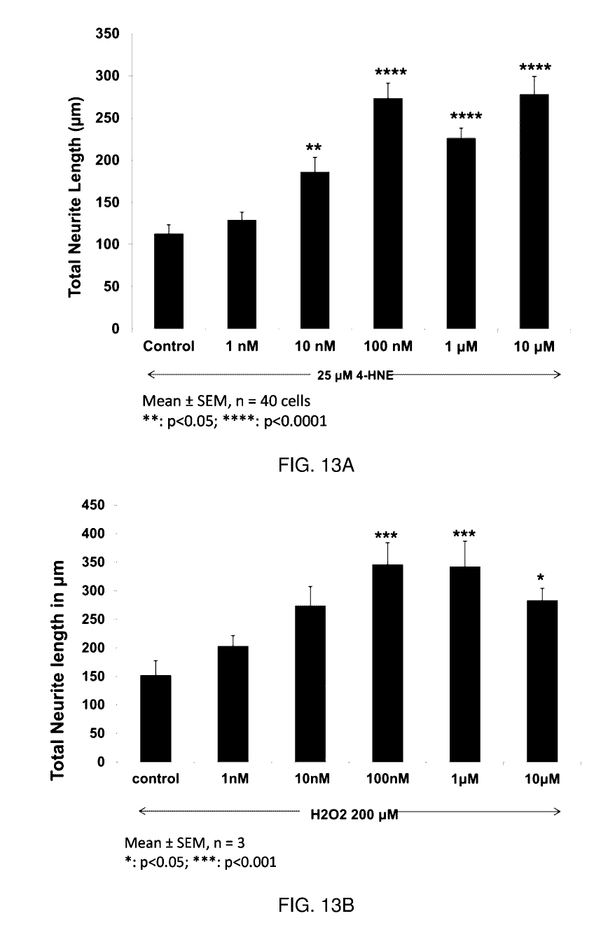

Mouse embryonic cortical neurons were treated with mock, 0, 0.001, 0.01, 0.1, 1, or 10 μM midi-GAGR for 2 days and then with 25 μM 4-HNE (FIG. 13A) or 200 μM H₂O₂ (FIG. 13B) for 1 day. After fixation, neurons were immunostained with anti-α-tubulin antibody. The total neurite length (μm) of cortical neurons was measured using Metamorph software. >10 nM Midi-GAGR still enhanced neurite outgrowth in cortical neurons in the presence of 25 μM 4-HNE (FIG. 13A) or 200 μM H₂O₂ (FIG. 13B).

Midi-GAGR enhances neuritogenesis and actin filopodia formation in differentiated neuro2A (N2A) cells.

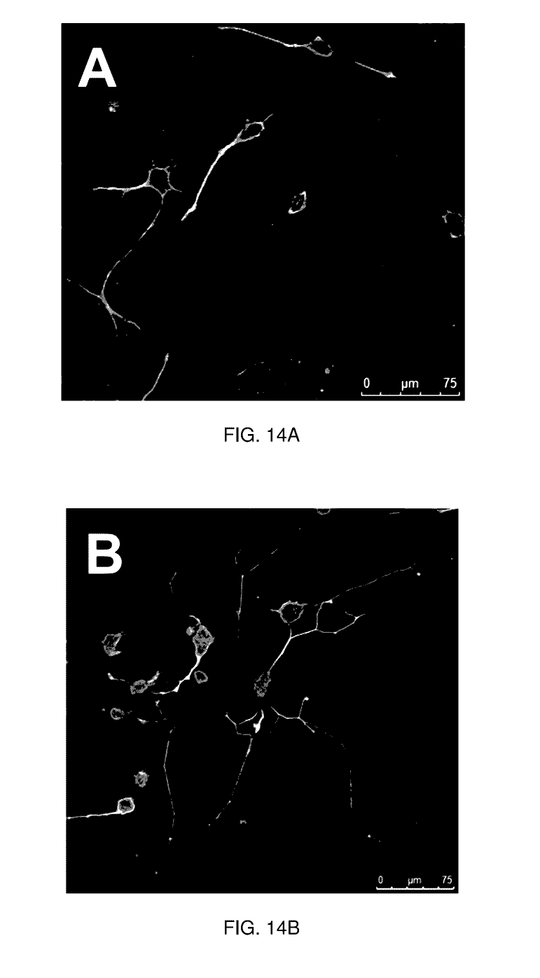

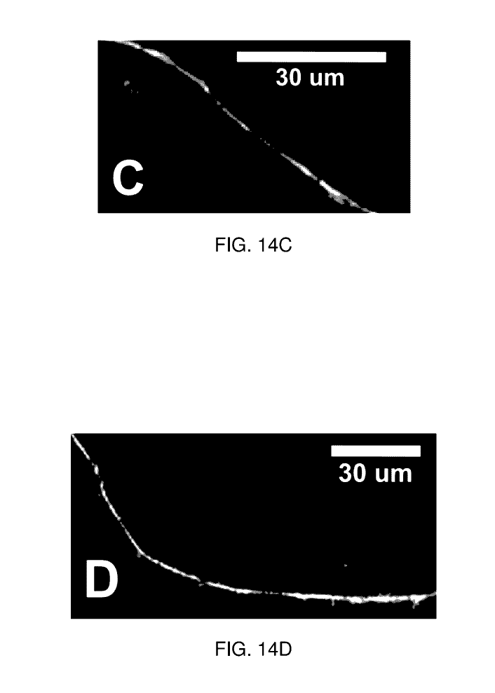

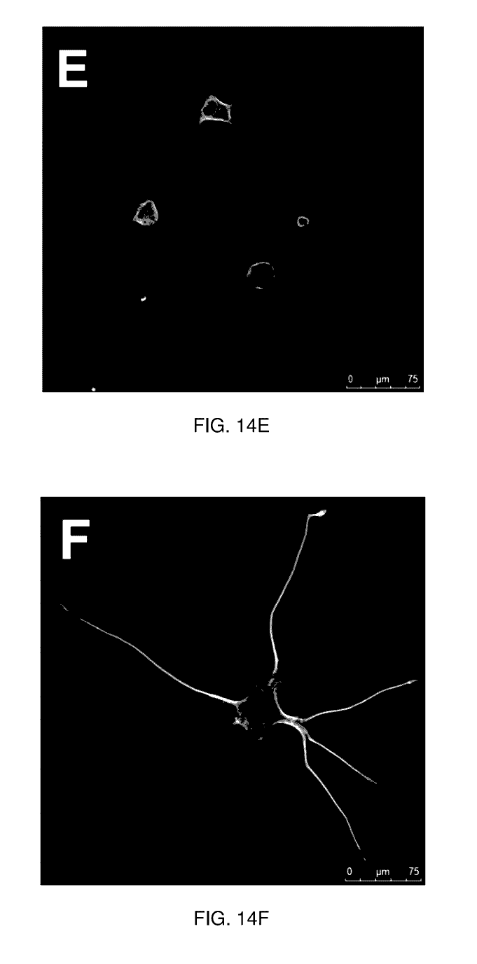

N2A cells were starved in serum-free medium for 3 days with mock (FIG. 14A) or 1 μM midi-GAGR (FIG. 14B), fixed, and immunostained with anti-α-tubulin antibody (green) and rhodamine-phalloidin (actin: red). The number and total length of neurites in differentiated N2A cells were measured. The number and total length of neurites in midi-GAGR-treated N2A cells were ˜1.7 fold higher than mock-treated cells. Midi-GAGR also increased formation of actin filopodia along neurites (FIG. 14D) while mock did not (FIG. 14C).

Midi-GAGR-treated N2A cells form neurites in the presence of 25 μM 4-HNE and 200 μM H₂O₂.

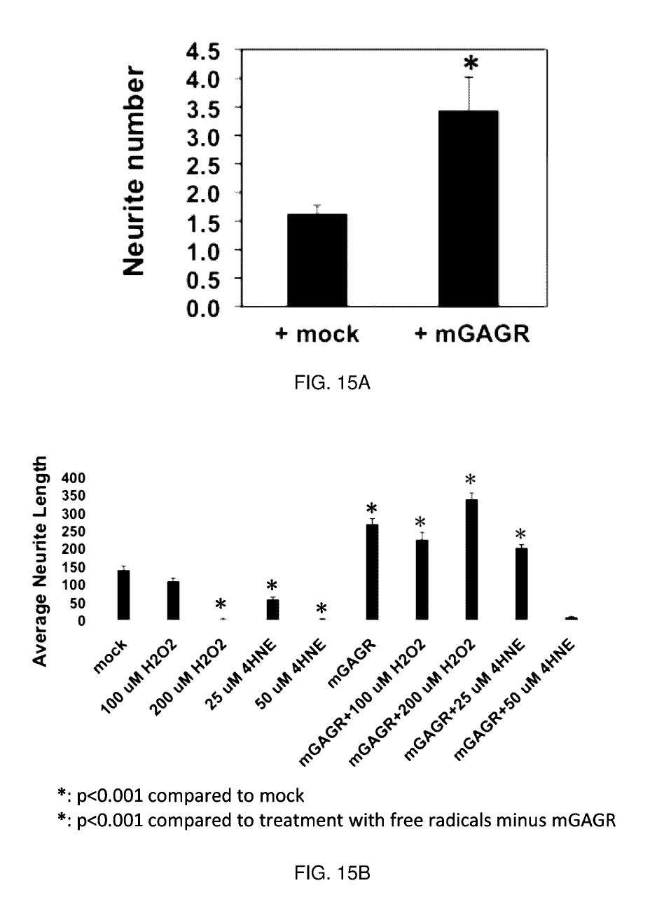

Following differentiation for 3 days with mock or 1 μM midi-GAGR, N2A cells were added with 10% FBS plus mock (FIG. 14E), 4-HNE (25 or 50 μM), or H₂O₂ (100 or 200 μM) (FIG. 14F: 200 μM H₂O₂) for 1 day. Cells were then fixed and immunostained with anti-α-tubulin antibody (green) and phalloidin (actin: red). The total neurite lengths in N2A cells were measured using Metamorph. Treatment with 1 μM midi-GAGR increased the number of neurites formed by N2A cells by ˜2 folds. The average total neurite length in mock-treated cells was decreased to ˜50% of mock treatment by 4-HNE (25 μM) and to ˜1% or mock by H₂O₂ (200 μM) while cells pretreated with 1 μM midi-GAGR maintained their neurites in the presence of 25 μM 4-HNE and 200 μM H₂O₂ (FIG. 15B). These data indicate that midi-GAGR prevents neurite atrophy caused by 4-HNE and H₂O₂.

Midi-GAGR Penetrates an In Vitro BBB Layer.

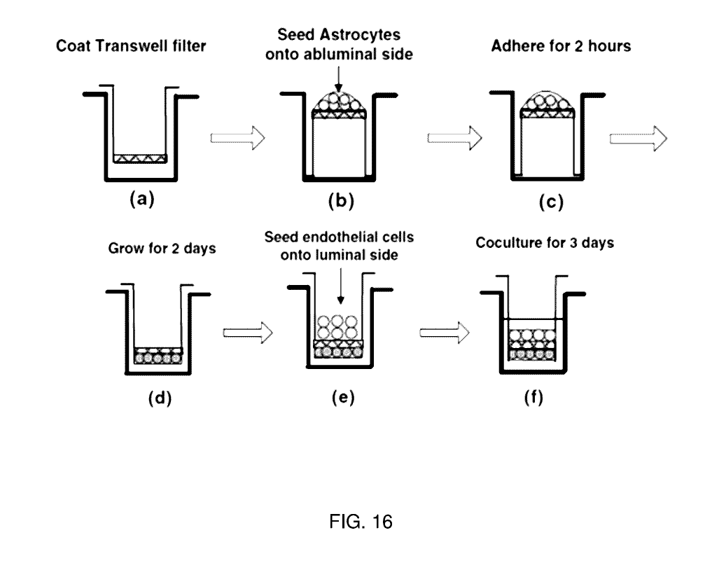

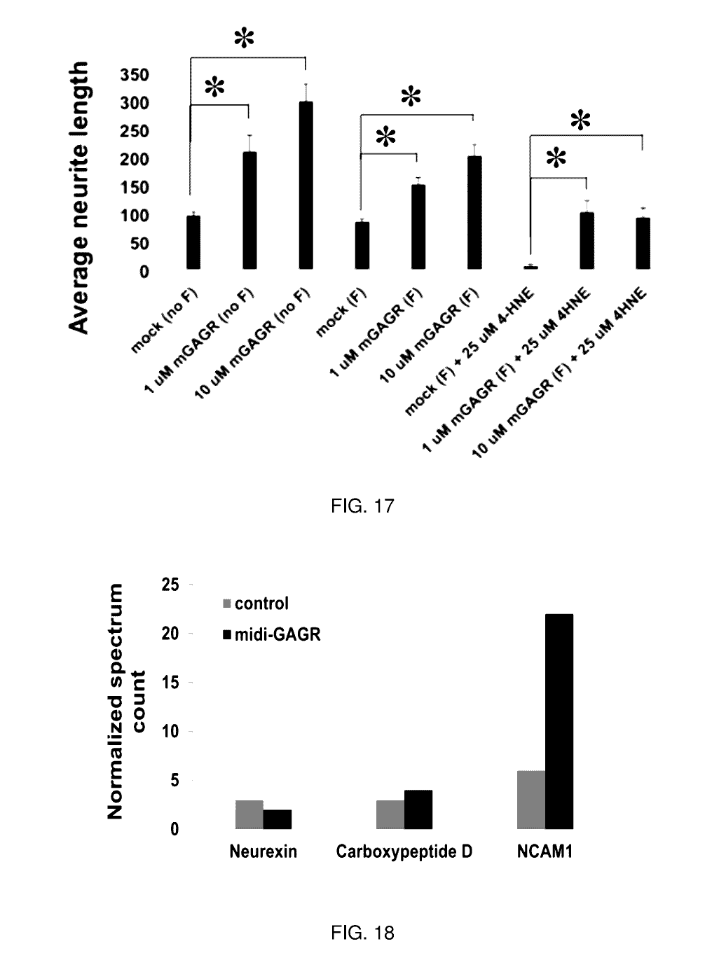

An in vitro blood-brain barrier (BBB) filter system was generated that consisted of bEND3 cells and astrocytes (FIG. 16). The filter system was placed on the top of each well of a 24-well plate, under which N2A cells were seeded on coverslips and incubated in serum-free DMEM. Mock, 1 or 10 μM midi-GAGR (mGAGR) was injected onto the top of the filter system. After 3-day differentiation, the filter systems were removed and N2A cells were treated with mock or 25 μM 4-HNE for 1 day. N2A cells that were under the filters into which 1 or 10 μM midi-GAGR was injected showed longer neurites than those injected with mock (FIG. 17). N2A cells that were under the filters into which 1 or 10 μM midi-GAGR was injected from long neurites even in the presence of 25 μM 4-HNE while cells injected with mock did not. This shows that midi-GAGR can penetrate the in vitro BBB filter system.

Midi-GAGR Binds to NCAM-180

A midi-GAGR-epoxy-sepharose 6B column (GE Healthcare Biosciences, Pittsburgh, Pa.) was used to purify midi-GAGR-interacting protein(s) from the plasma membrane fraction of mouse brain synaptosomes. Multiple purification experiments yielded the same result, showing the presence of a ˜180 kD protein only in the eluate from midi-GAGR beads. Mass spectrometry analysis showed that the 180 kD protein was NCAM-180 (FIG. 18). Activation of NCAM and FGFR induces MAPK pathways, resulting in activation of CREB. Midi-GAGR thus exerts its neurotrophic effect via its interaction with NCAM-180.

### Example 3

Described in this example is an efficient and effective method to track an exogenous polysaccharide among endogenous polysaccharides in animals. Instead of using complicated methods and equipment, a fluorescent tag, ANTS, can be used to label, track, and quantify a target polysaccharide in animal. 75% ethanol can be used to examine the structural intactness of ANTS-polysaccharide in animal sample. TCA and 75% ethanol can be used to separate free ANTS-polysaccharides from those bound to proteins. For example, the following equipment was used: SpectraMax M5 plate reader (Molecular Devices, Sunnyvale, Calif.); VERSA max plate reader (Molecular Devices); SoftMax Pro 5.2. (Molecular Devices); microcentrifuge.

Conjugation of ANTS to Polysaccharide

A 4.7 kD cleavage product of gellan gum (named midi-GAGR: digestion by α(1→3) glycosidase) was tagged with ANTS and examined regarding its BBB-permeability.

To conjugate ANTS (Molecular Probes, Eugene, Oreg.) to midi-GAGR, 45 μL of 7.4 mM midi-GAGR was mixed with 750 μL of 0.2 M ANTS (7.6 mg ANTS in 890 μL of acetic acid [3/17, v/v]), which gives the final ratio of 1:400 (polysaccharide:ANTS) that is optimal for high-efficiency conjugation between polysaccharide and ANTS.

The mixture was briefly vortexed and incubated in an 80° C. water bath for 30 min. The mixture was then added with 375 μL of 1 M NaCNBH₃ (Sigma-Aldrich, St. Louis, Mo.), briefly vortexed, and incubated in an 80° C. water bath for 90 min Although 37° C. can be also used for 15-h conjugation, 80° C. was used to shorten conjugation time. The mixture was split into 250-μL aliquots, each of which was mixed with 750 μL of 100% pure ethanol to make a final concentration of 75% ethanol.

The mixture was briefly vortexed, incubated at −80° C. for 30 min, and centrifuged at 3,000×g for 30 min to pellet ANTS-tagged midi-GAGR.

In order to remove free ANTS that might be trapped in ANTS-midi-GAGR pellet, the pellet was washed with 400 μL of 75% ethanol to dissolve ANTS trapped in the pellet. After the pellet of ANTS-midi-GAGR was resuspended in 400 μL of 75% ethanol by pipetting, it was re-precipitated at 3,000×g for 10 min. This step was repeated three times. The final pellet was resuspended in 50 μL of sterile de-ionized water.

Centrifugation at 15,700×g was also used for ethanol precipitation of ANTS-tagged polysaccharide. However, it resulted in precipitation of free ANTS. Therefore, the speed of centrifugation was decreased to 3,000×g, thus preventing the precipitation of free ANTS while still precipitating the similar amount of polysaccharide to that after the centrifugation at 15,700×g. In another embodiment, 70% ethanol can be used instead of 75% to reduce the precipitation of free ANTS that might be trapped in ANTS-polysaccharide pellet. Washes with 70% ethanol precipitation decreased the amount of ANTS at the pellet; however, there was also a noticeable loss of polysaccharide at the pellet after each wash. Thus, 75% ethanol was a desired concentration of ethanol to precipitate the maximal amount of polysaccharide and the minimal amount of free ANTS.)

Calculation of the Conjugation Ratio of ANTS to Polysaccharide in ANTS-Polysaccharide

The amounts of ANTS and midi-GAGR in ANTS-polysaccharide conjugate were measured by fluorometry and colorimetry, respectively, to calculate the ratio of ANTS to midi-GAGR in the conjugate.

For fluorometry, ANTS-polysaccharide pellet was resuspended and diluted in 50 of fresh water. The dilutions were placed in the wells of a 96-well black-wall plate. The emission fluorescence signals (excitation at 350 nm, emission at 520 nm; relative fluorescence units [RFUs]) of the dilutions were measured using SpectraMax M5 plate reader and SoftMax Pro 5.2. A standard curve of ANTS was generated using 0, 0.1, 0.3, 1, 3, and 10 mM ANTS to calculate the concentrations of ANTS in the samples.

For colorimetry, a modified phenol-sulfuric acid method modified was used.

ANTS-polysaccharide pellet was resuspended and diluted in 50 μL of fresh water. The dilutions were placed in the wells of a 96-well clear-wall plate. Each sample was added with 150 μL of concentrated H₂SO₄ and then 30 μL of 5% phenol (88% phenol liquefied USP [University of Toledo Medical Center, Toledo, Ohio] diluted in distilled water). The top of the plate was covered with a plate sealer and heated at 95° C. for 5 mM Using VERSA max plate reader and SoftMax Pro 5.4, the absorbance at 490 nm of each well was measured. A standard curve of midi-GAGR was generated using 0, 0.0074, 0.074, 0.74, and 7.4 mM midi-GAGR to calculate the concentrations of midi-GAGR in the samples.

Calculation of the Ratio of ANTS to Midi-GAGR in the Conjugate

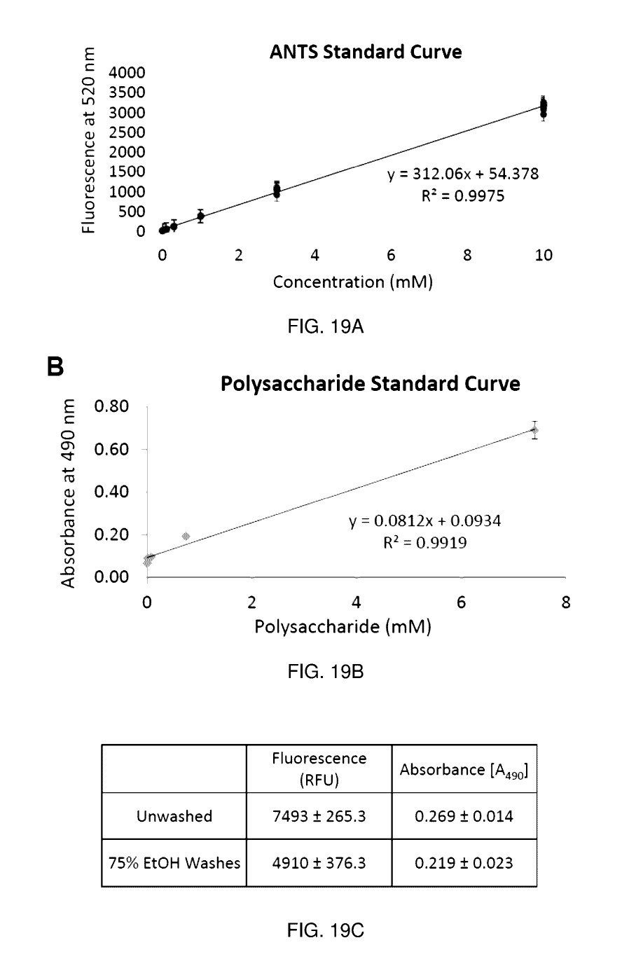

The standard curve of ANTS was generated using the RFUs of 0, 0.1, 0.3, 1, 3, and 10 mM free ANTS to quantify the concentrations of ANTS in the samples. FIG. 19A shows the standard curve of ANTS(R²=0.9975) that was generated on the basis of three different measurements.

The standard curve of midi-GAGR was generated using the absorbances at 490 nm for 0, 0.0074, 0.074, 0.74, and 7.4 mM free midi-GAGR. FIG. 19B shows the standard curve of midi-GAGR (R²=0.9919).

According to the standard curves, the RFU of ANTS-polysaccharide in the pellet before washes was ˜7493 and its absorbance at 490 nm was ˜0.269 (FIG. 19C).

After three washes, the mean RFU of ANTS-polysaccharide was significantly reduced to ˜4910 while the absorbance at 490 nm was only slightly reduced to 0.219. The values of RFU and absorbance at 490 nm were not further decreased by more washes after the third wash, showing that most of the loosely-associated free ANTS was removed from the pellet of ANTS-midi-GAGR.

According to the standard curves, the final pellet contained 16.18 mM ANTS and 1.55 mM midi-GAGR, which give the ratio of about 10:1 for ANTS to midi-GAGR.

It is to be noted that one ANTS was supposed to be conjugated to one reducing end of a polysaccharide, thus yielding the ratio of 1:1 for ANTS to midi-GAGR. However, more ANTS appeared to be conjugated to other hydroxyl groups on midi-GAGR, yielding the 10:1 ratio for ANTS to midi-GAGR. Polysaccharide was labeled with ANTS using EDC [1-Ethyl-3-3-dimethylaminopropyl carbodiimide] in order to conjugate the amino group of ANTS to the carboxyl group of glucuronic acid of midi-GAGR. However, the EDC conjugate of ANTS-midi-GAGR was not precipitated by 75% ethanol, showing that the EDC conjugate cannot be purified using 75% ethanol.)

Measurement of the Amount of ANTS-Polysaccharide that Enters the Brain and Blood Circulation

Administration and Measurement of ANTS-Midi-GAGR

To examine the BBB-permeability of ANTS-midi-GAGR, 40 μL, of 1 mM ANTS-midi-GAGR was administered into the nostril of Sprague-Dawley rats (male, 350-490 g, age of 8 weeks).

Rats were quickly anesthetized in an isoflurane induction chamber (isoflurane from Henry Schein Animal Health [Dublin, Ohio]). 5% isoflurane is administered into animal by a vaporizer with oxygen flowmeter (0.8-1.5 L/min) The percent of isoflurane was later adjusted to 2% until animal loses righting reflex.

20 μL of 1 mM ANTS-midi-GAGR was intra-nasally administered to each nostril. Animals were kept in the anesthetized condition for 5 mM after the administration of ANTS-polysaccharide to prevent the squirting-out of ANTS-polysaccharide from the noses.

At 6 h after the administration of ANTS-polysaccharide, animals were sacrificed using a guillotine. About 1 mL of trunk blood was collected in a vial immediately after decapitation. Trunk blood was incubated at room temperature for 1 h to coagulate and centrifuged at 3,000×g for 10 mM to remove the coagulated, which yields ˜400 μL, of serum. Simultaneously, the olfactory bulb tract and whole brain were also dissected out of the head of the decapitated animal. The brain and olfactory bulb tract were washed with 0.9% saline and homogenized in the equivalent volume of 1×PMEE buffer (pH 7.0; 35 mM KOH, 35 mM PIPES, 5 mM MgSO4, 1 mM EGTA, 1% BSA, and 0.5 mM EDTA) containing 1% Igepal CA-630 using a glass homogenizer (Wheaton, Millville, N.J.). The homogenized brain extract was centrifuged at 14,500×g for 20 mM and at 100,000×g for 30 mM to obtain brain cytosol. The amounts of ANTS-polysaccharide in the serum and the cytosols extracted from olfactory bulb and brain were measured by fluorometry.

Quantification of ANTS-Midi-GAGR in Brain and Blood

The amounts of ANTS-midi-GAGR in the samples were quantified using the standard curve of ANTS and the conjugation ratio of 10:1 for ANTS to midi-GAGR (FIGS. 19A-19B).

The RFUs of the cytosols extracted from the olfactory bulb tracts of all rats were lower than ˜50 RFUs that were the same as the basal fluorescence units of those of rats administered with saline alone (data not shown), thus showing that little ANTS-midi-GAGR was routed to the olfactory bulb tract.

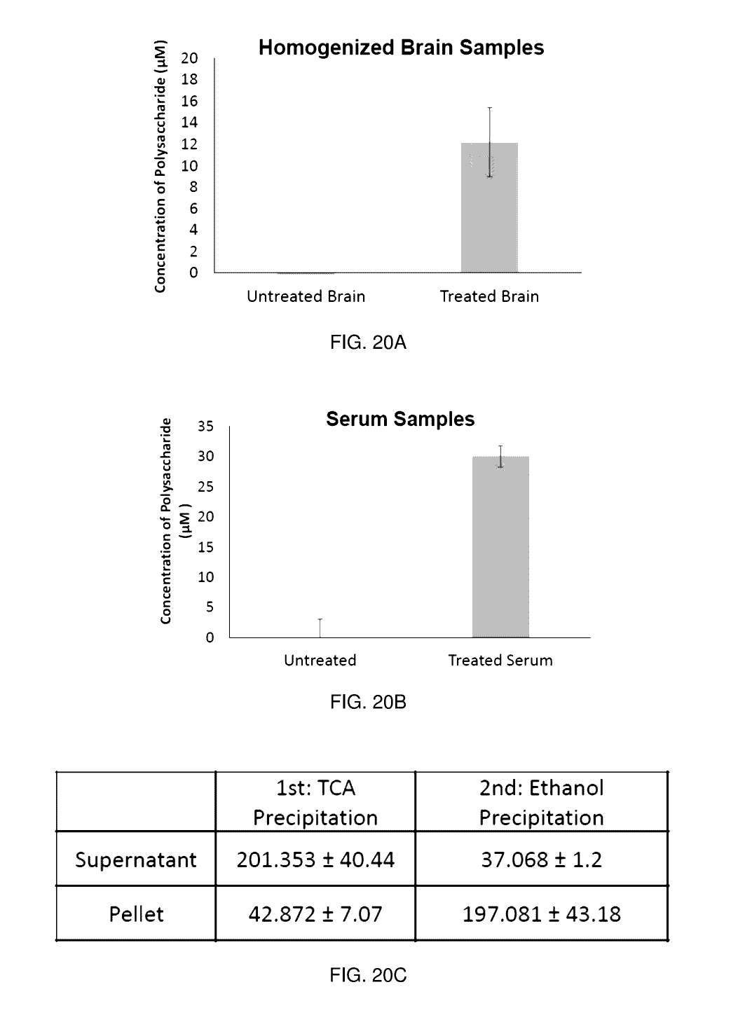

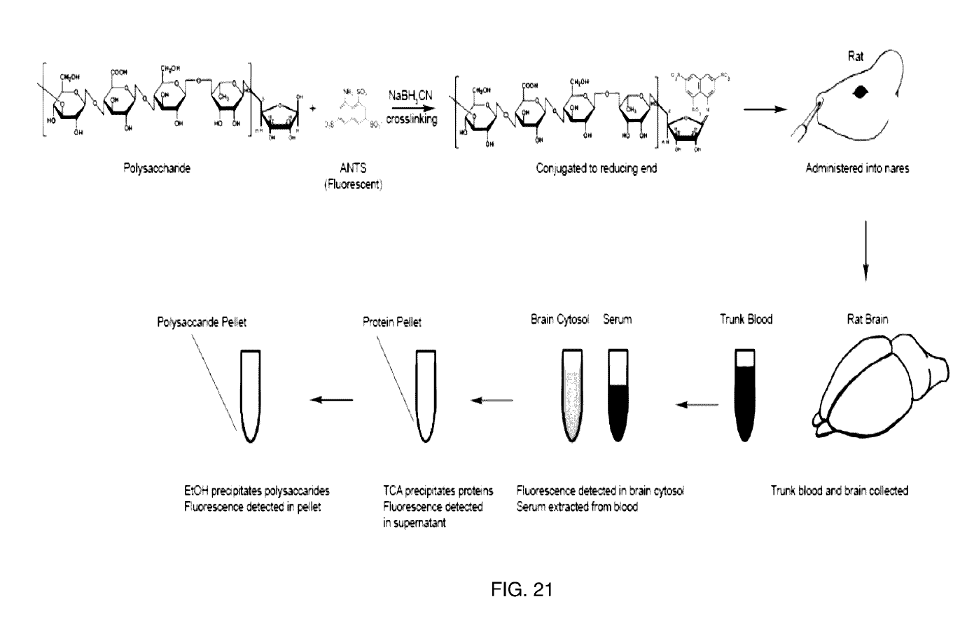

The RFUs of the sera and brain cytosols were significantly above 50 RFUs. The RFU values of brain cytosols and sera were converted to the concentrations of midi-GAGR using the ratio of 10:1 for ANTS to midi-GAGR. The brain cytosols and sera of the rats administered with ANTS-midi-GAGR contained ˜12 μM (FIG. 20A) and ˜28 μM (FIG. 20B), respectively, of midi-GAGR. Thus, intra-nasally administered midi-GAGR can enter the brain and blood circulation.

Examination of the Structural Intactness of ANTS-Midi-GAGR in the Serum

It was possible that the fluorescence of the sera was emitted from free ANTS that might be generated by the cleavage of ANTS-midi-GAGR during the circulation in the blood. Therefore, it was examined whether ANTS-midi-GAGR in the sera was structurally intact or not by 75% ethanol precipitation that only precipitate ANTS-midi-GAGR but not free ANTS.

100 μL of the supernatant was added with 300 μL of 100% ethanol to make the final concentration of 75% ethanol and centrifuged at 3,000×g to precipitate serum polysaccharides including ANTS-midi-GAGR, leaving polysaccharide-free ANTS in the supernatant. The pellet was resuspended in the equivalent volume of water to that of the supernatant. The RFUs of the supernatant and pellet resuspension were measured by fluorometry. The RFUs of the supernatant fell down to below 50 while those of the pellet resuspension were close to those of the supernatant before ethanol precipitation. This shows that most of ANTS-midi-GAGR was structurally intact in the serum.

Examination of the Binding of ANTS-Midi-GAGR to Serum Protein

Also examined was whether ANTS-midi-GAGR in the serum bound to serum proteins like albumin or not. TCA was used to precipitate all the proteins and protein-bound molecules including polysaccharides but leave protein-free polysaccharides in the supernatant.

100 μL of serum sample in a microtube was added with 10 μL of TCA to make the final concentration of 10% TCA, incubated at 4° C. for 10 min, and centrifuged at 15,700×g for 5 min. The supernatant over the pellet was transferred to a new tube and the pellet containing proteins was resuspended in 100 μL of water. The RFU of the pellet resuspension was measured by fluorometry. The RFUs of the pellet resuspension was below the basal fluorescent units (50 RFUs) while those of the supernatants still yielded ˜201 RFUs (FIG. 20C), thus showing that little ANTS-polysaccharide was precipitated along with serum proteins.

100 μL of the supernatant was added with 300 μL of 100% ethanol to make the final concentration of 75% ethanol and centrifuged at 3,000×g to precipitate serum polysaccharides including ANTS-midi-GAGR. The pellet was resuspended in 50 μL water. The RFUs of the supernatant and pellet resuspension were measured by fluorometry. The RFU of the supernatant was below the basal fluorescent units (50 RFUs) while the pellet resuspension yielded ˜201 RFUs, thus showing that most ANTS-midi-GAGR remained intact in the supernatant after TCA precipitation.

ANTS-midi-GAGR that enters the brain and blood circulation does maintain its intact structure inside animal and does not bind to serum protein for 6 h after its intra-nasal administration. Given that the cerebrospinal fluid should contain less digestive enzymes than the peripheral blood, it is now shown herein that ANTS-midi-GAGR in the brain should be intact as well.)

### Example 4

### Further Examples

### Therapeutic/Prophylactic Methods and Compositions

Further described herein are methods of treatment and prophylaxis by administration to a subject an effective amount of a therapeutic compound, i.e., mini- and/or midi-GAGR. In a preferred aspect, the therapeutic is substantially purified. The subject is preferably an animal, including but not limited to, animals such as cows, pigs, chickens, etc., and is preferably a mammal, and most preferably human.

Various delivery systems are useful to administer a therapeutic compound, e.g., encapsulation in liposomes, microparticles, microcapsules, etc. Methods of introduction include, but are not limited to, intradermal, intramuscular, intraperitoneal, intravenous, subcutaneous, intranasal, and oral routes. The therapeutic compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the therapeutic compositions locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers.

### Example 5

### Peptide Nanotubes for Slow Release of Drugs (Such as Midi-GAGR) at Peripheral Nerve Injury

To examine the neurogenic effect of midi-GAGR/mini-GAGR on the peripheral nervous system, an animal model of peripheral nerve cut was used. Left infraorbital nerve transactions were used to cut the middle of axons that originate from the cell bodies of the trigeminal ganglia under the eyes and innervate into the sensory nerve endings linked to the whiskers of rat. Then, midi-GAGR or mini-GAGR were injected into the site of peripheral nerve cut. If midi-GAGR or mini-GAGR exerts its neurogenic effect, the axons will regenerate from the cell bodies of the trigeminal ganglia and re-innervate into the whiskers. Horseradish peroxidase (HRP, retrograde marker) was injected to the nerve endings underneath the whiskers and, if axons regenerate, is retro-transported back to the cell bodies of the trigeminal ganglia. Peptide nanotubes (PNT) were used for the controlled and slow release of midi-GAGR or mini-GAGR from the injection site to the surrounding areas.

Adult female Sprague-Dawley rats (250-350 g; 2-2.5 months old, bred-in-house) were used for this example. A total of 30 animals (5 animals per group for 4 week study and 10 animals per group for 8 week study) were housed in groups of two at room temperature under a 12 h light/dark cycle. Food and water were provided ad libitum. All the procedures of animal use outlined in this example were approved by the Animal Care and Use Committee of University of Toledo College of Medicine and Life Science in accordance with National Institutes of Health guidelines.

Peptide nanotubes (PNTs) were engineered for encapsulation of midi-GAGR or mini-GAGR for controlled slow release from the injection site. For the encapsulation, diphenylalanine peptide monomer (Bachem, Torrance, Calif.) was dissolved in a highly volatile solvent HFP (1,1,1,3,3,3-hexafluoro-2-propanol) at a concentration of 100 mg/mL. Subsequently, monomer solution was diluted to 2 mg/mL in sterile distilled water for the self-assembly of PNTs. The PNTs were then completely dried using a rotary evaporator at 50° C. For encapsulation, dried PNTs (4 mg) were incubated with 100 μM midi-GAGR or mini-GAGR solution in PBS or PBS alone at 4° C. on a rotator for 3 days. PNTs incubated with PBS alone were used as control. After 3 days, PNTs were separated from solution by ultracentrifugation at 15,000×g for 10 min Most supernatant except for 40 μL above the PNT pellet was removed and the pellet was resuspended in the remaining solution.

Left infraorbital nerve transaction was used to cut the middle of axons of trigeminal ganglia neurons. Sprague Dawley female rats were anesthetized with intraperitoneal injection of Ketamine (50 mg/kg)/Xylaine (15 mg/kg) solution. The hair on the top of the head was shaved and the rat was mounted on a stereotaxic frame. All surgeries were performed in a sterile condition under a surgical microscope. The skin incision was made at the midline of the head. Underlying fascia and muscle were blunt dissected with a pair of cotton-coated forceps and the orbit was gently retracted to reveal the fascicles of infraorbital nerve (ION). The ION can be seen lying deep within the orbital cavity, lying in the infraorbital bony fissure. The fascicles of the infraorbital nerve (ION) lying deep within the infraorbital bony fissure at the left side were cut by iridectomy scissors. ION at the right side was uncut as a control. Following exposure, the left ION was cut with iridectomy scissors.

After bleeding was stopped, 40 μL of nanotubes that encapsulate either midi-GAGR or PBS were applied on the sites where the nerve cut was made. The overlying muscle and skin were then clipped back and the animals were allowed to recover for 4 weeks. After 4 or 8 weeks, animals were anesthetized with a mixture of ketamine (50 mg/kg, I.P.) and xylazine (15 mg/kg, I.P.). Facial hair was removed using depilatory cream to expose whisker follicles. 40% solution of HRP (Worthington Biochemical Corp. Lakewood, N.J.) in 2% dimethylsulfoxide was injected into four whisker follicles (A1, A2, A3 and A4), 20 μL each. Control nanotube-treated animals were injected with HRP on both sides (cut and non-cut), whereas midi-GAGR nanotube-treated animals were injected with HRP on ION cut side only. Right side (non-cut ION) labeling worked as a positive control.

Three days after the labeling, rats were killed using CO₂ and perfused transcardially with saline followed by fixation with 4% paraformaldehyde. Animals were perfused transcardially with buffered saline followed by fixation with TMB fixative (1% paraformaldehyde and 2.5% glutaraldehyde in PBS). Left trigeminal ganglions were dissected out and post-fixed in cold TMB fixative for 18-36 h at 4° C. In some animals, right trigeminal ganglions were also dissected out to serve as positive controls. The next day, ganglions were placed horizontally and cut into 20 μm thick serial sections on a freezing microtome. All the sections were pre-incubated for 20 min in 3,3′ diaminobenzidine tetrahydrochloride in acetate buffer. After 20 minutes, hydrogen peroxide dilutant was added to the above mixture and sections were incubated for another 20 min on a shaker at room temperature. Sections were mounted on coverslips using permount and examined by light microscopy.

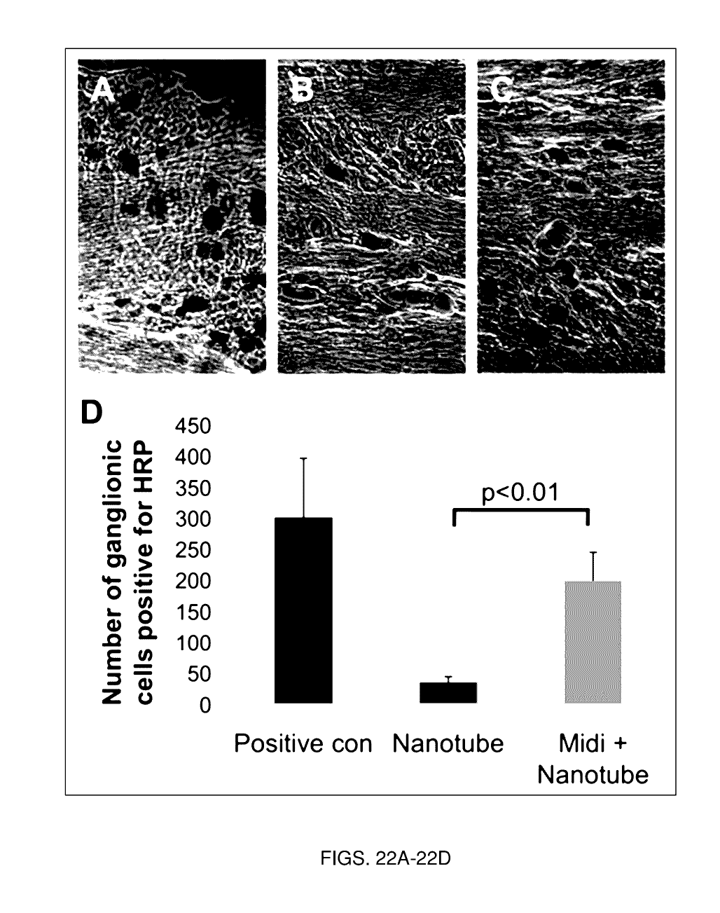

HRP-labeled nerve cells were identified by the presence of small dark granules in the cytoplasm and counted using a light microscope. Rats injected with nanotubes alone showed few positive HRP stains in the cell bodies of trigeminal ganglia compared to uncut control (FIGS. 22A-22D). In contrast, about 70% of axons were regenerated in rats injected with nanotubes containing midi-GAGR compared to uncut control. Thus, it is clear that midi-GAGR (and therefore also mini-GAGR) can regenerate the severed axons of peripheral neurons. The PNTs used for slow drug release make multiple injections to the damaged peripheral nerve unnecessary.

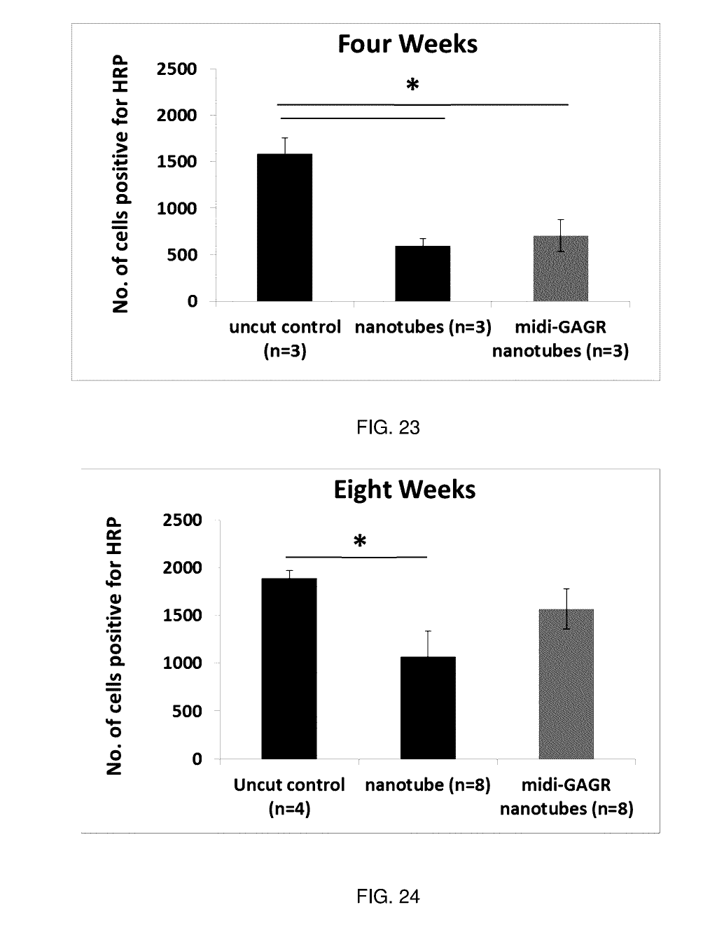

After 4 weeks, retrograde tracer (HRP) was applied to whisker follicles to examine infraorbital nerve re-innervation to vibrissae. The method allows detection of cell bodies in trigeminal ganglia corresponding to nerve fibers that regrows and innervates back to whisker follicles, A1-A4. All the sections of trigeminal ganglia labelled with HRP were counted using light microscopy. The labeling method could detect 1586±176 cells in un-cut control animals. Compared to un-cut controls, post-cut animals applied with control nano-tubes showed significant reduction in number of cells labeled with HRP in trigeminal ganglia after 4 weeks (598±72). (FIG. 23.) This indicates that the infraorbital nerve was successfully transected and nanotube alone could not enhance regeneration of nerve back to whisker pad.

The total number of HRP labeled cell bodies in left trigeminal ganglia from nanotube- or midi-GAGR nanotube-treated animals were counted using light microscopy after 8 weeks. The right trigeminal ganglia from nanotube-treated animals were also dissected and used as an un-cut positive control. Light microscopy results revealed that animals applied with control nanotubes post-nerve transection had significantly fewer HRP-labeled cells compared to un-cut controls after 8 weeks. This indicates that the control nanotube application could not increase regeneration of infraorbital nerve. On the other hand, midi-GAGR-encapsulated nanotube group showed an increase in the number of HRP labeled cells after 8 weeks.

This example demonstrates the neuro-regenerative ability of the neurotrophic agent midi-GAGR using an infraorbital nerve transection model in adult rats. This example shows the in vivo evidence for the neurotrophic effects of midi-GAGR. These results indicate that midi-GAGR-encapsulated nanotubes can enhance re-innervation of transected nerve back to vibrissae pad of rats in 8 weeks. Also, the number of HRP labeled cells in midi-treated group did not differ from un-cut control group (uncut control vs. midi-GAGR nanotube; 1888.75±80 vs. 1570.5±208.4; ns). Midi-GAGR-encapsulated nanotubes were able to almost fully regenerate transected nerves back to their target within 8 weeks.

Peptide nanotubes (PNTs) were used as a carrier to slowly release midi-GAGR over the study period. In this example, the nanotube alone group did not significantly differ from midi-nanotube group at 4 weeks or 8 weeks. Thus, it is possible that the nanotube itself provides a conduit for nerve-regeneration and midi-GAGR act as a neurotrophic agent to enhance nerve growth.

To summarize, this example provides in vivo evidence for the neuro-regenerative capacity of midi-GAGR following complete nerve transection. Given that nanotubes provided controlled release of midi-GAGR over the study period, midi-GAGR-encapsulated nanotubes can be used as a neurotrophic agent following peripheral nerve injury to induce nerve regeneration.

Pharmaceutical compositions.

Such compositions comprise a therapeutically effective amount of a therapeutic, such as mini- and/or midi-GAGR, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In one embodiment, the composition is formulated in accordance with routine procedures as a pharmaceutical composition adapted for intravenous administration to human beings. For example, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutic formulation can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic formulation which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems

Kits

Also provided are pharmaceutical packs or kits comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

Any of the compositions described herein may be comprised in a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial.

When the components of the kit are provided in one and/or more liquid solutions, the liquid solution is an aqueous solution, with a sterile aqueous solution being one preferred solution.

However, the components of the kit may be provided as dried powder(s). When components are provided as a dry powder, the powder can be reconstituted by the addition of a suitable solvent. It is envisioned that the solvent may also be provided in another container means. It may also include components that preserve or maintain the polysaccharides mini- and midi-GAGR, or that protect against their degradation.

The compounds, methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A composition comprising nanotubes assembled from a monomer solution comprising a diphenylalanine peptide, wherein a digestion product of low acyl gellan gum (LA-GAGR) is encapsulated in the nanotubes.

2. The composition of claim 1, wherein the digestion product comprises midi-GAGR or mini-GAGR.

3. The composition of claim 1, wherein the diphenylalanine peptide is dissolved in 1,1,1,3,3,3-hexafluoro-2-propanol.

4. The composition of claim 1, wherein the monomer solution is present in water at a concentration of about 2 mg/mL.

5. A method of inducing nerve regeneration and/or treating peripheral nerve injury, the method comprising:
administering an effective amount of midi-GAGR or mini-GAGR to a subject in need thereof to induce nerve regeneration or treat peripheral nerve injury.

6. The method of claim 5, wherein the method comprises encapsulating midi-GAGR in peptide nanotubes to produce midi-GAGR-encapsulated nanotubes, and administering an effective amount of the midi-GAGR-encapsulated nanotubes to the subject to induce nerve regeneration.

7. The method of claim 6, wherein the peptide nanotubes comprise a diphenylalanine peptide dissolved in 1,1,1,3,3,3-hexafluoro-2-propanol.

8. The method of claim 6, wherein the administration is via sustained release of the midi-GAGR from the midi-GAGR-encapsulated nanotubes.

9. The method of claim 5, wherein the method comprises encapsulating mini-GAGR in peptide nanotubes to produce mini-GAGR-encapsulated nanotubes, and administering an effective amount of the mini-GAGR-encapsulated nanotubes to the subject to induce nerve regeneration.

10. The method of claim 9, wherein the peptide nanotubes comprise a diphenylalanine peptide dissolved in 1,1,1,3,3,3-hexafluoro-2-propanol.

11. The method of claim 9, wherein the administering is via sustained release of the mini-GAGR from the mini-GAGR-encapsulated nanotubes.

12. A method of making peptide nanotubes comprising:
dissolving a diphenylalanine peptide monomer in a highly volatile solvent to produce a monomer solution;
diluting the monomer solution in water to cause self-assembly of peptide nanotubes; and
drying the peptide nanotubes.

13. The method of claim 12, wherein the highly volatile solvent comprises 1,1,1,3,3,3-hexafluoro-2-propanol.

14. The method of claim 12, wherein the peptide nanotubes are encapsulated with a digestion product of low acyl gellan gum (LA-GAGR).

15. The product of the method of claim 12.
